(19) United States
(12) Patent Application Publication
(10) Pub. No.: US 2023/0123911 A1
(43) Pub. Date: Apr. 20, 2023

(54) MODULATION OF IMMUNE CELLS

(71) Applicant: OCTAGON THERAPEUTICS, INC., CAMBRIDGE, MA (US)

(72) Inventors: Nathaniel SHERDEN, BOSTON, MA (US); SHEN YU, WINCHESTER, MA (US); LUIS CASTILLO-MENENDEZ, CHELSEA, MA (US); ISAAC STONER, SOMERVILLE, MA (US); BRENT CEZAIRLIYAN, BOSTON, MA (US)

(21) Appl. No.: 17/759,775

(22) PCT Filed: Jan. 29, 2021
(86) PCT No.: PCT/US2021/015891

§ 371 Date: Jul. 29, 2022

Related U.S. Application Data
(60) Provisional application No. 62/969,530, filed on Feb. 3, 2020.
(60) Provisional application No. 62/968,667, filed on Jan. 31, 2020.

(51) Int. Cl. A61K 47/54; A61K 45/06

(52) U.S. Cl. CPC A61K 47/549; A61K 47/548; A61K 45/06

(57) ABSTRACT

Disclosed are immune cell-selective small molecule IMPDH inhibitor compounds and methods of their synthesis and use to treat proliferative disorders.

### CROSS REFERENCE TO RELATED APPLICATIONS

This application claims priority to United States provisional application nos. 62/968,667, filed Jan. 31, 2020, and 62/969,530, filed Feb. 3, 2020, entitled “Modulation of Immune Cells”, the contents of each of which are incorporated by reference herein in its entirety.

### FIELD OF THE INVENTION

The present invention is in the fields of medicine and proliferative diseases. More particularly, the invention relates to small molecule cell-specific inhibitors of immune cell metabolic enzymes for the treatment of cancer, atherosclerosis, and autoimmune disorders.

### BACKGROUND OF THE INVENTION

Proliferative diseases (PDs)” is a unified term to describe several disease types where excessive proliferation of cells and turnover of cellular matrix contribute significantly to the pathogenesis. PDs encompass cancer, inflammatory diseases such as atherosclerosis (AS), and many autoimmune diseases such as rheumatoid arthritis (RA), inflammatory bowel diseases (IBD), psoriasis as well as infectious diseases of bacterial, viral or fungal nature. As such, PDs affects a vast number of populations. For example, autoimmune disease affects 3% to 5% of the population. There are more than 100 distinct autoimmune disorders; the majority involve multiple genetic and environmental factors. The concordance of autoimmune disease in identical twins is 12% to 67%, highlighting the complexity of underlying disease mechanisms and the potential importance of stochastic or epigenetic phenomena.

The first generation therapies for PDs that demonstrated clinical utility were antiproliferative agents such as cancer chemotherapy agents and immunosuppressants. These compounds non-selectively hit fast proliferating cells, usually by interfering cellular metabolisms, and thereby exhibit a range of toxicities including bone marrow suppression, GI-toxicity (hitting intestine epithelial cells or crypt cells, and hair loss (hitting hair follicles). Importantly, fast-proliferating lymphocytes are important for normal immune function and is critical in controlling infection and in cancer surveillance. Non-selective suppression of lymphocytes exposes patients to elevated risk of opportunistic infections as well as neoplasia.

Biologic therapies based on highly selective monoclonal antibodies that modify specific inflammatory or effector pathways have autoimmune disorders e.g, TNF inhibitors, IL-6 inhibitor, IL-12 inhibitor, BLyS inhibitor CTLA4-IgGs and anti-CD20 antibodies).

Unfortunately, many of these biologics have significant drawbacks. For example, the anti-CD20 antibody, Rituximab (Rituxan), widely used in B-cell malignancy treatment as well as autoimmune diseases, triggers cell death via antibody-dependent cellular cytotoxicity (ADCC) when it binds to CD20 on a B-cell surface it However, Rituximab is known to cause headache and back pain, in addition to possessing a slow administration infusion rate (50 mg/hr), which can take up to eight hours. In addition, its administration increases the risk of infections and malignancies as it depletes B-cells; normal B-cell functions are essentially absent for patients treated with Rituximab. Treatments like Intravenous Immune Globulin (IVIG) can partially restore B-cell functions, but the method has severe toxicity liabilities.

In autoimmune disorders like RA, anti-TN F biologic drugs such as Adalimumab (Humira, also used in other autoimmune disorders such as IBDs and psoriasis) are often used as front-line treatment. Unfortunately, anti-TNT biologics generally suffer from low response rate, elevated risk for tumors and infections and pain at injection site. For these reasons, it is often necessary to combine biologic drugs with other small molecule chemotherapeutic agents or disease-modifying antirheumatic drugs (DMARDs) to achieve satisfactory therapeutic effects, examples include combination of Rituximab with four chemotherapeutic agents (known as the R—CHOP regimen, Rituxirmab with cyclophosphamide, hydroxydaunorubicin, oncovin, prednisone/prednisolone) in treatment of non-Hodgkin lymphomas, and combination of adalimumab with methotrexate in treatment of RA, However, when combining with these non-selective small molecule drugs, the advantage of cell-selectivity of the antibodies is lost.

Mycophenolate mofetil (MMF, Cellcept) was developed as an immunosuppressant and is widely used to prevent allograft rejection and is used off-label in a variety of autoimmune diseases. MMF is a prodrug of mycophenolic acid (MPA) which increases the bioavailability of MPA and inhibits IMPDH. However, MMF suffers from poor PK properties and significant GI toxicity. Several other IMPDH inhibitors have been developed as anti-proliferative agents for select autoimmunity and cancer indications (the Cortellis database), but were abandoned late in clinical development due to off-target safety concerns.

Thus, improved small molecule antiproliferative drugs that are disease-modifying and immune cell-type-selective are highly desired.

### SUMMARY OF THE INVENTION

It has been discovered that certain metabolic enzymes such as IMPDH are selectively and highly expressed in specific immune cell populations. It has also been discovered that certain novel IMPDH inhibitors can be chemically optimized for guidance to and selection for certain immune cell types. These inhibitors show a high level of immunomodulatory and anti-proliferative effects in cell-based assays.

This discovery has been exploited to develop the present disclosure, which, in part, is directed to immune cell-selective small molecule inhibitor compounds of IMPDH (“CSIIs” or “CSII compounds”) and methods of their synthesis and use to treat proliferative disorders such as cancer, atherosclerosis and autoimmune disorders.

In an aspect, provided herem is a CSII compound of Formula I:

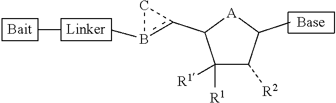

or a pharmaceutically acceptable salt thereof, wherein:
---- is an optional bond;
A is selected from the group consisting of CH₂, NH, O, and S;

B is selected from the group consisting of C, CH, CH₂, N, NH, O, and S;

C is selected from the group consisting of CH₂, NH, O, and S;

R¹ is selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{1′} is selected from the group consisting; of OH, SH, NH₂, N₃, and halo;
R² is selected from the group consisting of OH, SH, NH₂, N₃, and halo:
Base is selected from the group consisting of

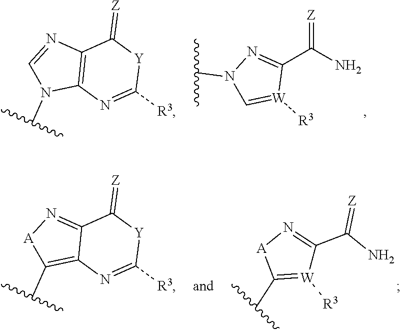

wherein:
Z is selected from the group consisting of O, NH, and S;
Y is selected from the group consisting of O, NH, and S,
W is selected from the group consisting of CH, CH₂, NH, and N;

R³ is selected from the group consisting of OH, SH, NH₂, N₃, and halo:
Linker is selected from the group consisting of

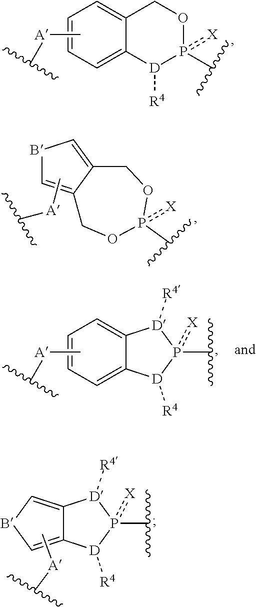

wherein:
A′ is independently, at each occurrence, selected from the group consisting of CH₂, NH, O, S, CO₃, CO₂N, CO₂S, and CO₂N;
B′ is independently, at each occurrence selected from the group consisting of CH₂, NH, O, and S;
D and D′ are each independently selected from the group consisting; of O, N, NH, and S;
X is independently, at each occurrence, selected from the group consisting of O, NH, and S;
R⁴ and R^{4′} are each independently selected from the group consisting of H, C₁₋₄ alkyl, C₂₋₄ alkenyl, C₁₋₄ n vinyl, and C₀₋₄ allyl, all of which are optionally substituted with one, two, three, or four halo;
Bait is an oligomer comprising 1-5 units, which are independently, at each occurrence, selected from the group consisting of

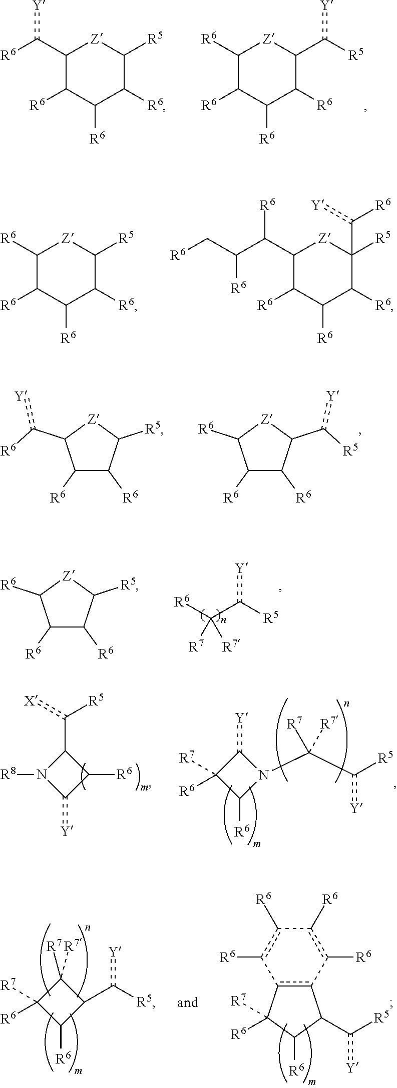

wherein:
Z′ is independently, at each occurrence, selected from the group consisting of CH₂, NH, O, and S;
X′ and Y′ are independently, at each occurrence, selected from the group consisting of O, NH, and S;
R⁶ is independently, at each occurrence, selected from the group consisting of a bond to any R⁶, a bond to any R⁸, and a bond to A′;
R⁶ is independently, at each occurrence, selected from the group consisting of a bond to R⁵, H, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{c}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;

R⁷ and R^{7′} are each independently, at each occurrence, selected from the group consisting of H, NH₂, OH, C₆₋₁₀ aryl, 5-10 membered heteroaryl, C₁₋₄ alkyl, —(CH₂)₁₋₃C₆₋₁₀ aryl, and —(CH₂)₁₋₃-5-10 membered heteroaryl, wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D},—R^{A}—(SO₂)—R^{C}, —R^{A} (SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 23, or 4; and
n is 0, 1, 2, or 3.
   In an embodiment of Formula I,

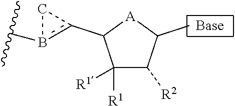

is a payload.

In some embodiments, the compound of Formula I is a compound of Formula Ia:

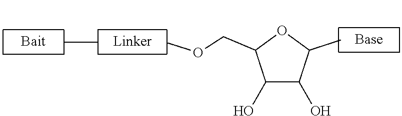

or a pharmaceutically acceptable salt thereof.

In others embodiment, the compound of Formula I is a compound of Formula Ib:

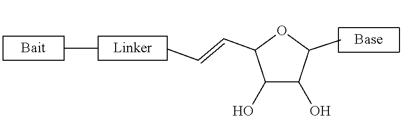

or a pharmaceutically acceptable salt thereof.

In some embodiments, the Base comprises

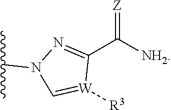

In other embodiments, the Base comprises

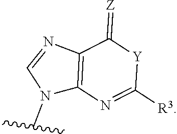

In another embodiment, the Base comprises

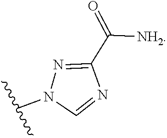 [SMILES: *n1cnc(C(N)=O)n1]

In some embodiments, the Base comprises

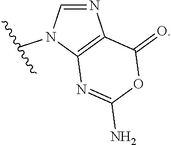 [SMILES: *n1cnc2c(=O)oc(N)nc21]

In some embodiments, the Linker comprises

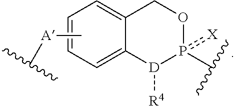

In another embodiment, the Linker comprises

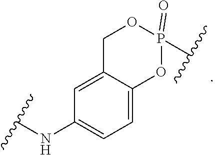 [SMILES: *Nc1ccc2c(c1)COP(*)(=O)O2]

In still another embodiment, the Linker comprises

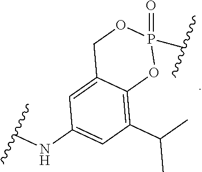 [SMILES: *Nc1cc2c(c(C(C)C)c1)OP(*)(=O)OC2]

In yet another embodiment, the Linker comprises

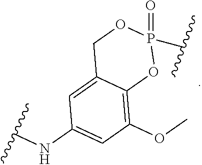 [SMILES: *Nc1cc2c(c(OC)c1)OP(*)(=O)OC2]

In another embodiment, the Linker comprises

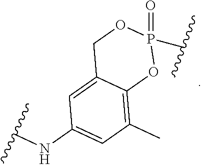 [SMILES: *Nc1cc(C)c2c(c1)COP(*)(=O)O2]

In certain embodiments, the Bait is a linear or is a branched oligomer.

In some embodiments, the compound of Formula I is selected from the group consisting of

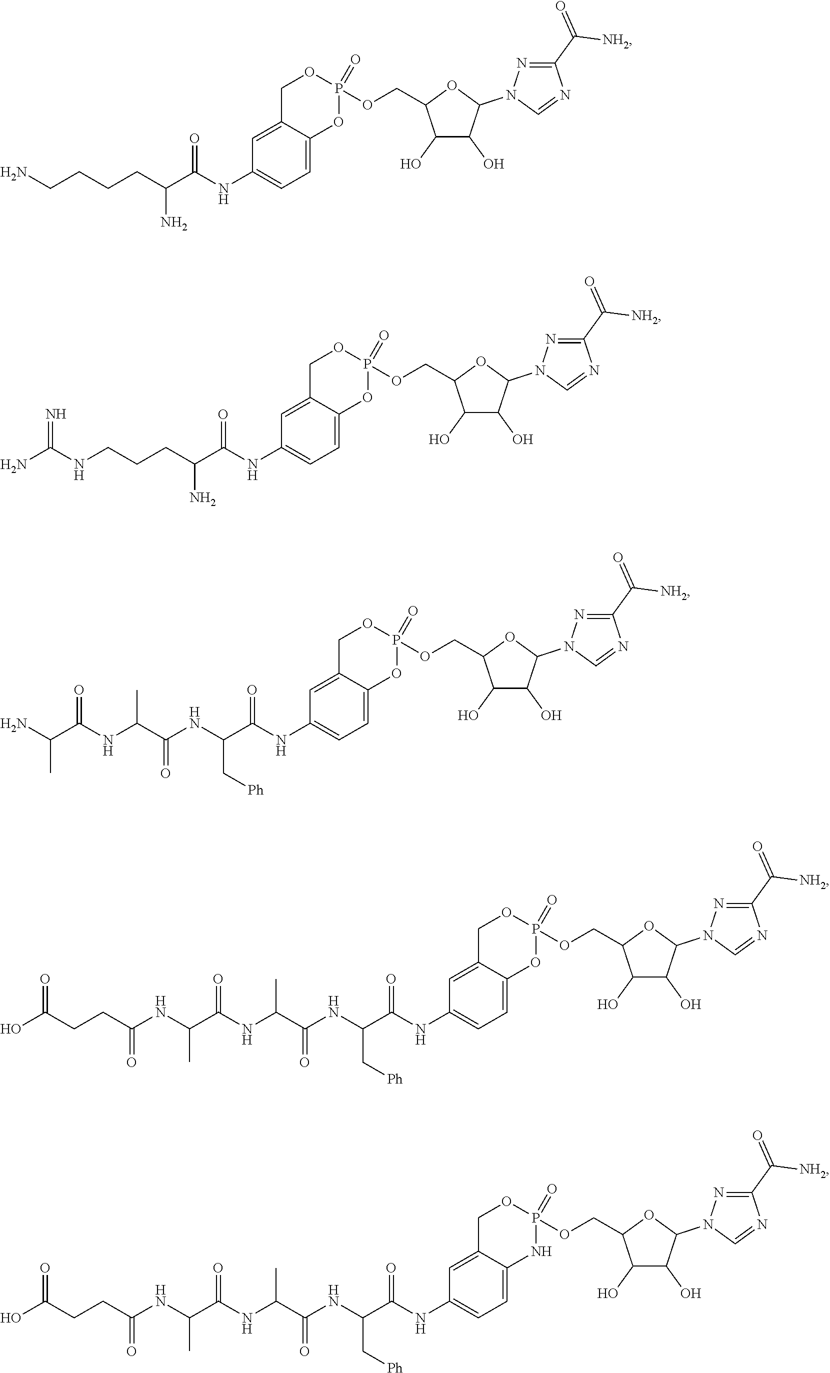 [SMILES: NCCCCC(N)C(=O)Nc1ccc2c(c1)COP(=O)(OCC1OC(n3cnc(C(N)=O)n3)C(O)C1O)O2 ; N=C(N)NCCCC(N)C(=O)Nc1ccc2c(c1)COP(=O)(OCC1OC(n3cnc(C(N)=O)n3)C(O)C1O)O2 ; CC(N)C(=O)NC(C)C(=O)NC(Cc1ccccc1)C(=O)Nc1ccc2c(c1)COP(=O)(OCC1OC(n3cnc(C(N)=O)n3)C(O)C1O)O2 ; CC(NC(=O)CCC(=O)O)C(=O)NC(C)C(=O)NC(Cc1ccccc1)C(=O)Nc1ccc2c(c1)COP(=O)(OCC1OC(n3cnc(C(N)=O)n3)C(O)C1O)O2 ; CC(NC(=O)CCC(=O)O)C(=O)NC(C)C(=O)NC(Cc1ccccc1)C(=O)Nc1ccc2c(c1)COP(=O)(OCC1OC(n3cnc(C(N)=O)n3)C(O)C1O)N2]

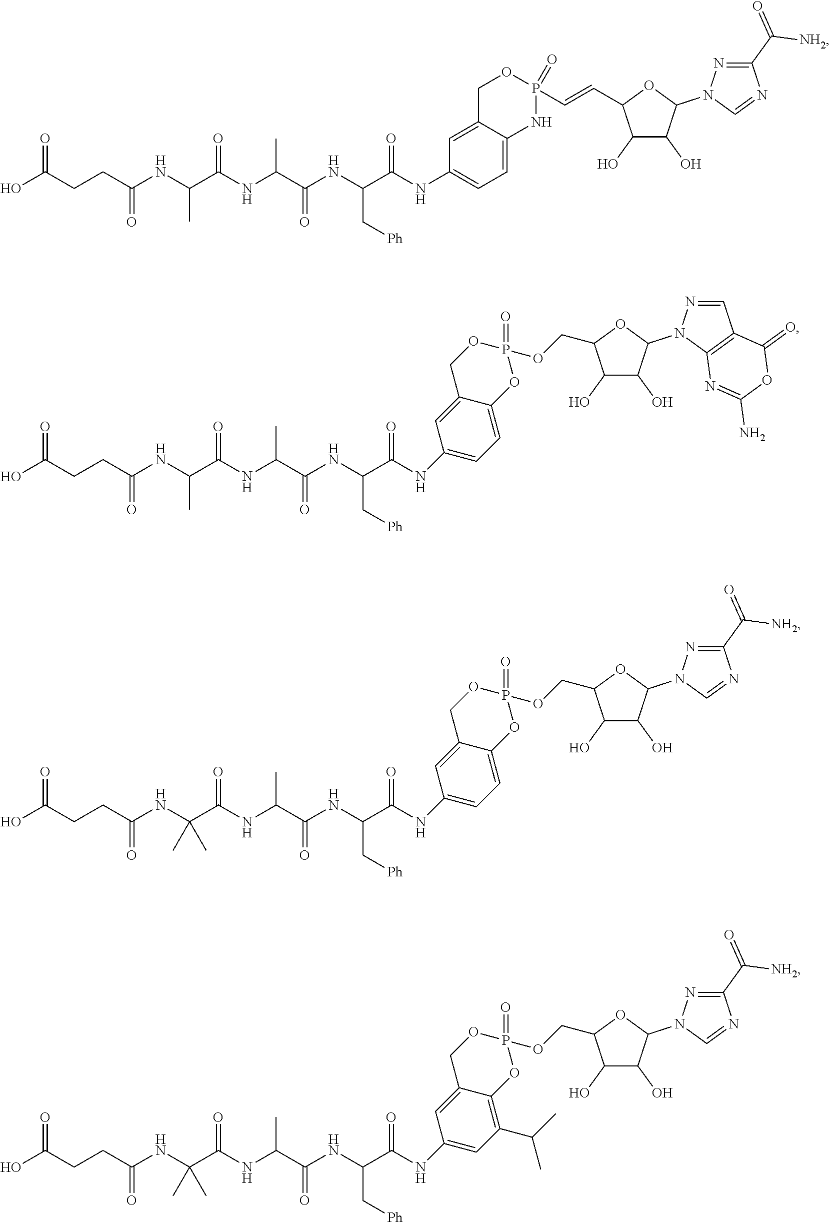 [SMILES: CC(NC(=O)CCC(=O)O)C(=O)NC(C)C(=O)NC(Cc1ccccc1)C(=O)Nc1ccc2c(c1)COP(=O)(/C=C/C1OC(n3cnc(C(N)=O)n3)C(O)C1O)N2 ; CC(NC(=O)CCC(=O)O)C(=O)NC(C)C(=O)NC(Cc1ccccc1)C(=O)Nc1ccc2c(c1)COP(=O)(OCC1OC(n3ncc4c(=O)oc(N)nc43)C(O)C1O)O2 ; CC(NC(=O)C(C)(C)NC(=O)CCC(=O)O)C(=O)NC(Cc1ccccc1)C(=O)Nc1ccc2c(c1)COP(=O)(OCC1OC(n3cnc(C(N)=O)n3)C(O)C1O)O2 ; CC(NC(=O)C(C)(C)NC(=O)CCC(=O)O)C(=O)NC(Cc1ccccc1)C(=O)Nc1cc2c(c(C(C)C)c1)OP(=O)(OCC1OC(n3cnc(C(N)=O)n3)C(O)C1O)OC2]

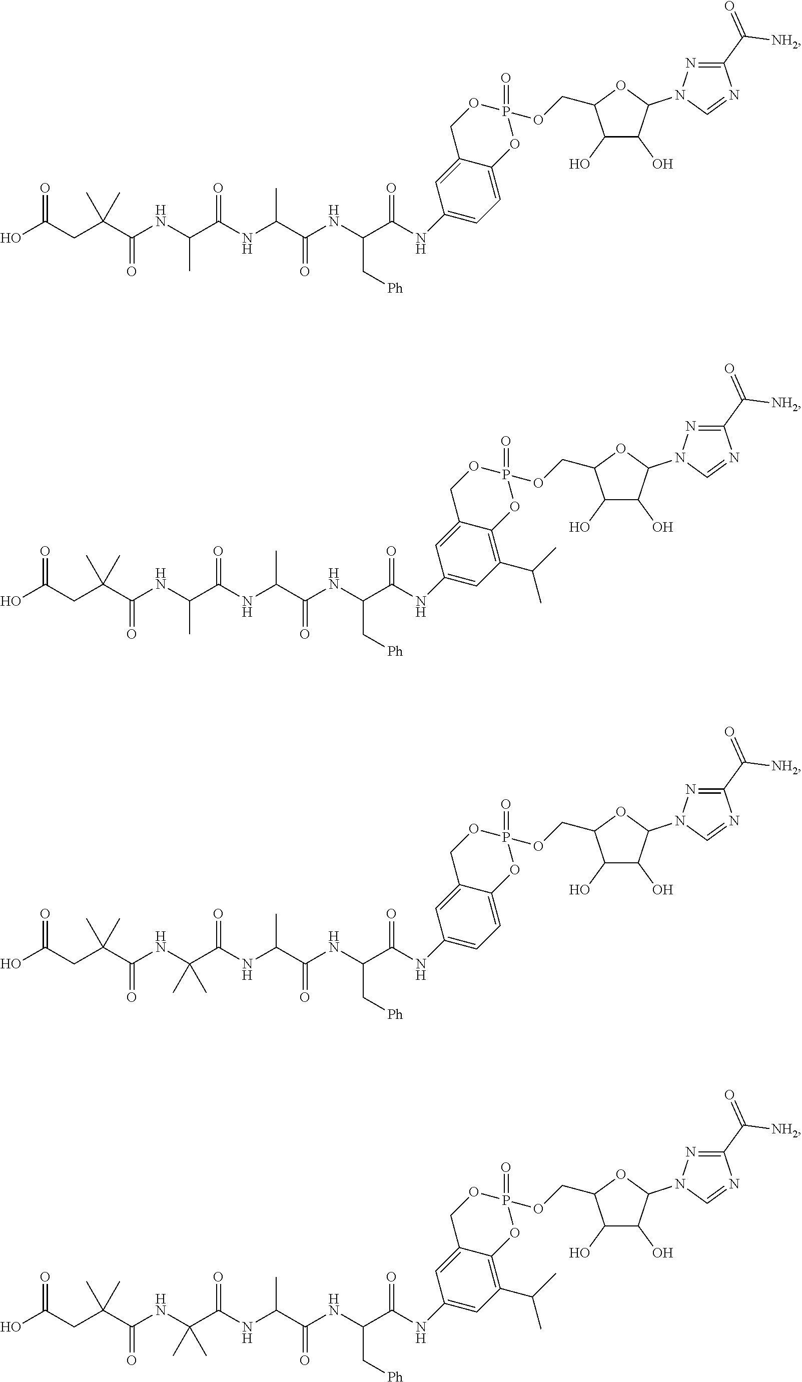 [SMILES: CC(NC(=O)C(C)NC(=O)C(C)(C)CC(=O)O)C(=O)NC(Cc1ccccc1)C(=O)Nc1ccc2c(c1)COP(=O)(OCC1OC(n3cnc(C(N)=O)n3)C(O)C1O)O2 ; CC(NC(=O)C(C)NC(=O)C(C)(C)CC(=O)O)C(=O)NC(Cc1ccccc1)C(=O)Nc1cc2c(c(C(C)C)c1)OP(=O)(OCC1OC(n3cnc(C(N)=O)n3)C(O)C1O)OC2 ; CC(NC(=O)C(C)(C)NC(=O)C(C)(C)CC(=O)O)C(=O)NC(Cc1ccccc1)C(=O)Nc1ccc2c(c1)COP(=O)(OCC1OC(n3cnc(C(N)=O)n3)C(O)C1O)O2 ; CC(NC(=O)C(C)(C)NC(=O)C(C)(C)CC(=O)O)C(=O)NC(Cc1ccccc1)C(=O)Nc1cc2c(c(C(C)C)c1)OP(=O)(OCC1OC(n3cnc(C(N)=O)n3)C(O)C1O)OC2]

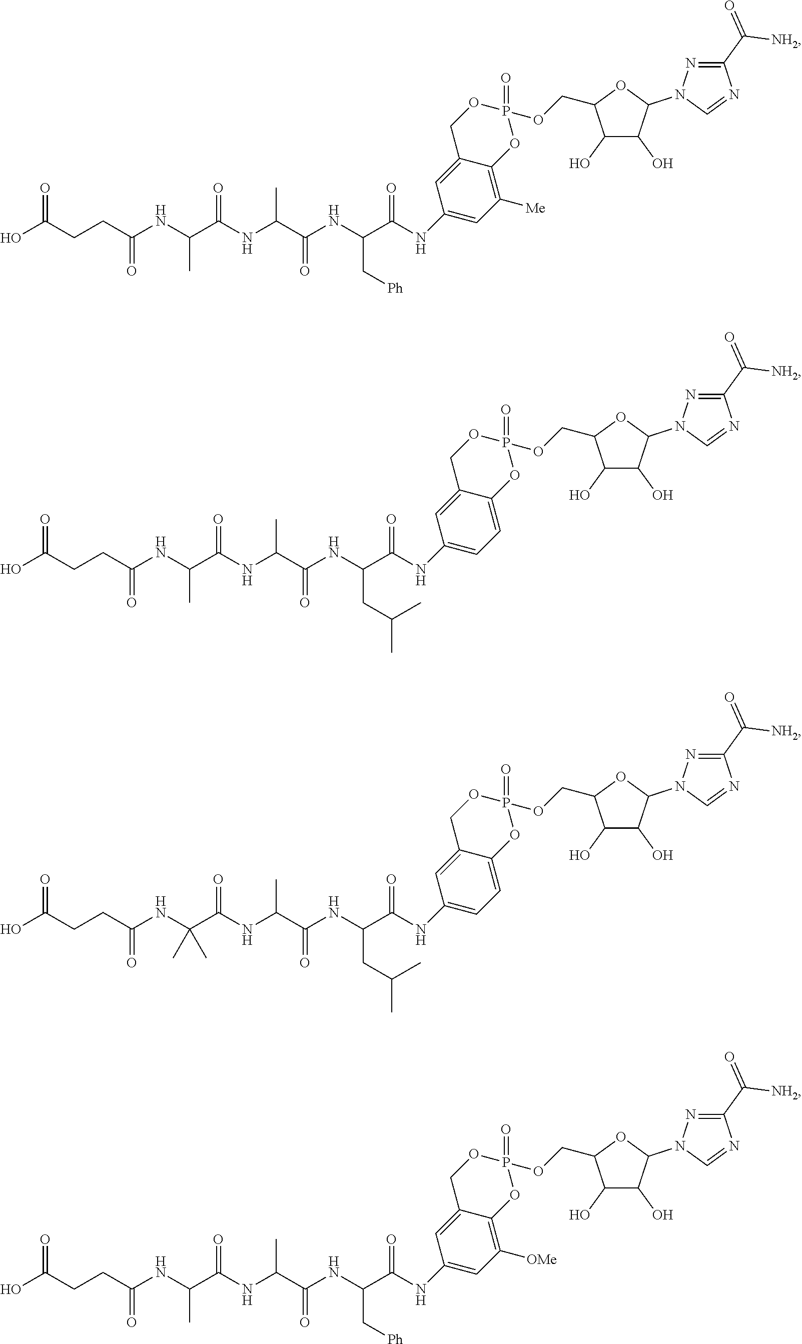 [SMILES: Cc1cc(NC(=O)C(Cc2ccccc2)NC(=O)C(C)NC(=O)C(C)NC(=O)CCC(=O)O)cc2c1OP(=O)(OCC1OC(n3cnc(C(N)=O)n3)C(O)C1O)OC2 ; CC(C)CC(NC(=O)C(C)NC(=O)C(C)NC(=O)CCC(=O)O)C(=O)Nc1ccc2c(c1)COP(=O)(OCC1OC(n3cnc(C(N)=O)n3)C(O)C1O)O2 ; CC(C)CC(NC(=O)C(C)NC(=O)C(C)(C)NC(=O)CCC(=O)O)C(=O)Nc1ccc2c(c1)COP(=O)(OCC1OC(n3cnc(C(N)=O)n3)C(O)C1O)O2 ; COc1cc(NC(=O)C(Cc2ccccc2)NC(=O)C(C)NC(=O)C(C)NC(=O)CCC(=O)O)cc2c1OP(=O)(OCC1OC(n3cnc(C(N)=O)n3)C(O)C1O)OC2]

and a pharmaceutically acceptable salt thereof.

In yet other embodiments, the compound of Formula I is a compound of Formula Ic

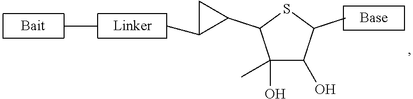

or a pharmaceutically acceptable salt thereof.

In some embodiments, the Base comprises

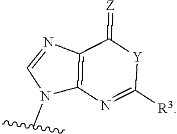

In other embodiments, the Base comprises

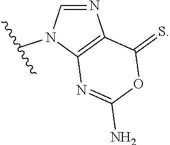 [SMILES: *n1cnc2c(=S)oc(N)nc21]

In some embodiments, the Linker comprises

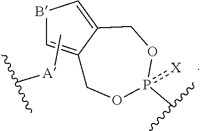

In other embodiments, the Linker comprises

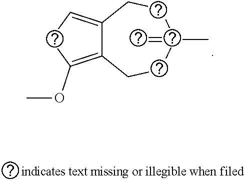

In certain embodiments, the compound of Formula I is selected from the group Consisting of

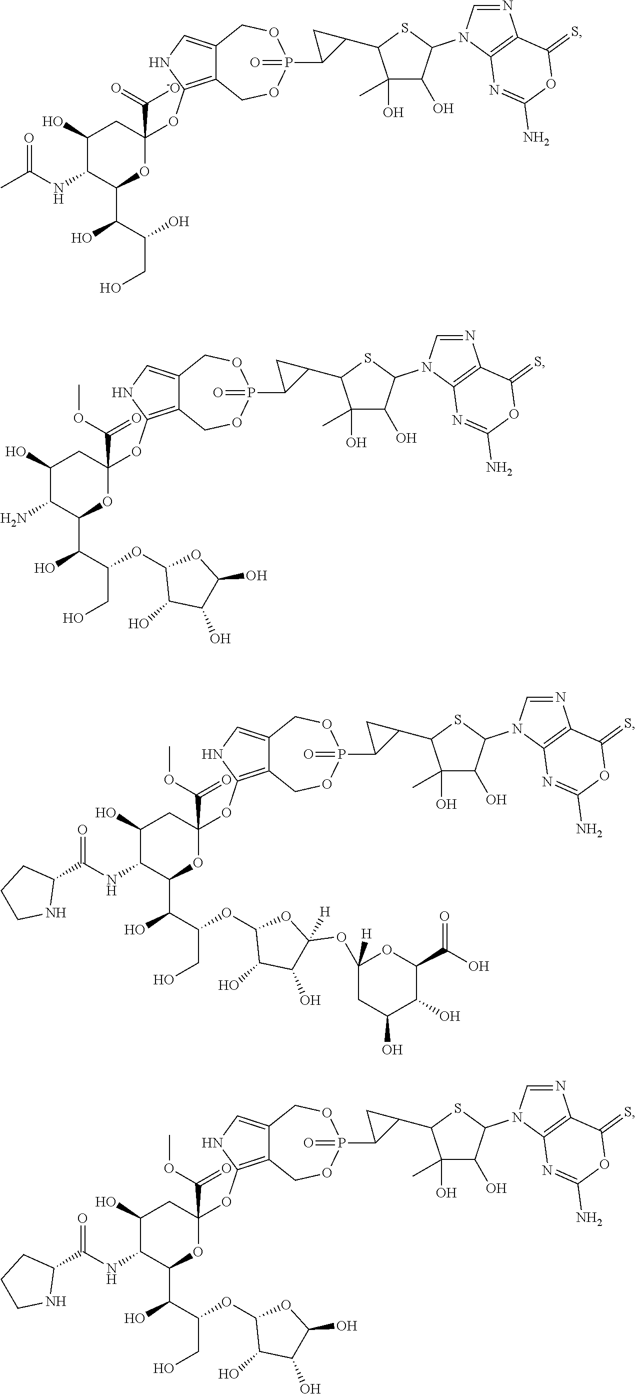

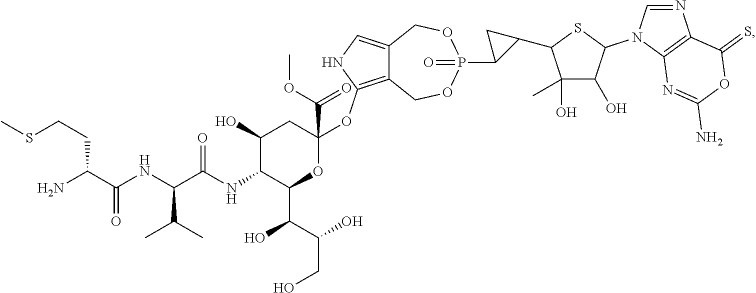

an a pharmaceutically acceptable salt thereof.

In another aspect, provided herein is a CSII compound of Formula II

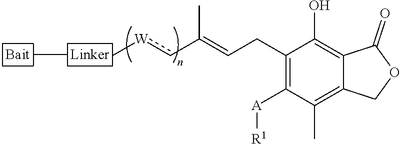

or a pharmaceutically acceptable salt thereof, wherein:
A is selected from the group consisting of CH₂, NH, O, and S;
W is independently, at each occurrence, selected from the group consisting of CH, CH₂, NH, and N;
r is 1, 2, or 3;
Linker is selected from the group consisting of

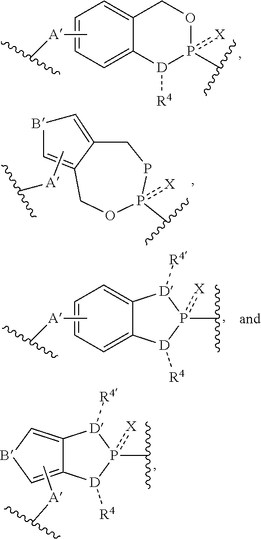

wherein:
A′ is independently, at each occurrence, selected from the group consisting of CH₂, NH, O, S, CO₃, CO₂N, CO₂S, and CO₂N;
B′ is independently, at each occurrence, selected from the group consisting of CH₂, NH, O, and S;
D and D′ are each independently selected from the group consisting of O, NH, and S;
X is selected from the group consisting of O, NH, and S; and
R⁴ and R^{4′} are each independently selected from the group consisting of H, C₁₋₄ alkyl, C₂₋₄ alkenyl, C₂₋₄ vinyl, and C₃₋₄ allyl, all of which are optionally substituted with one, two, three, or four halo:
Bait is an oligomer comprising 1-5 units, which is, independently at each occurrence, selected from the group consisting of

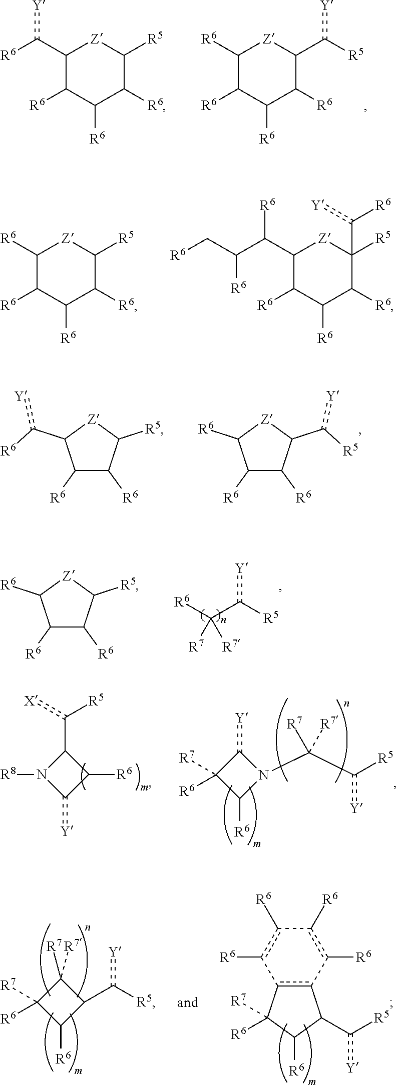

wherein:
Z′ is independently at each occurrence, selected from the group consisting of CH₂, NH, O, and S;
X′ and Y′ are independently, at each occurrence selected from the group consisting of O, NH, and S;
R⁵ is independently, at each occurrence, selected from the group consisting of a bond to any R⁶, a bond to any R⁸, and a bond to A′;
R⁶ is independently, at each occurrence, selected from the group consisting of a bond to R⁵, H, ═O, ═NH, ═S, —R^{A}—(C ═R^{B})—R^{C}, —R^{A}—(C═R^{B})—R^{A}—R^{C}, R^{A}—(SO₂)—R^{D}, —R^{A}—(SO₂) R^{c}, and —R^{A}—(SO₂)—R^{A}—R^{t}
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1, 2, or 3.

In an embodiment of Formula II,

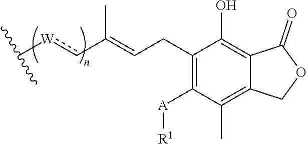

### is a Payload.

In some embodiments, the compound of Formula II is a compound of Formula IIa:

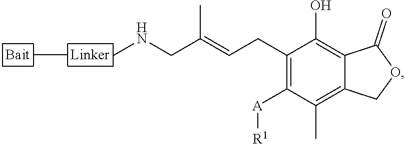

or a pharmaceutically acceptable salt thereof.

In some embodiments of the compound the Linker comprises:

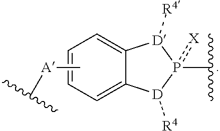

In other embodiments, the Linker comprises:

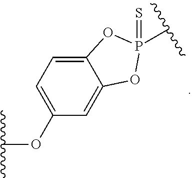 [SMILES: *Oc1ccc2c(c1)OP(*)(=S)O2]

In certain embodiments, the compound of Formula II is selected from the group consisting of

and a pharmaceutically acceptable salt thereof.

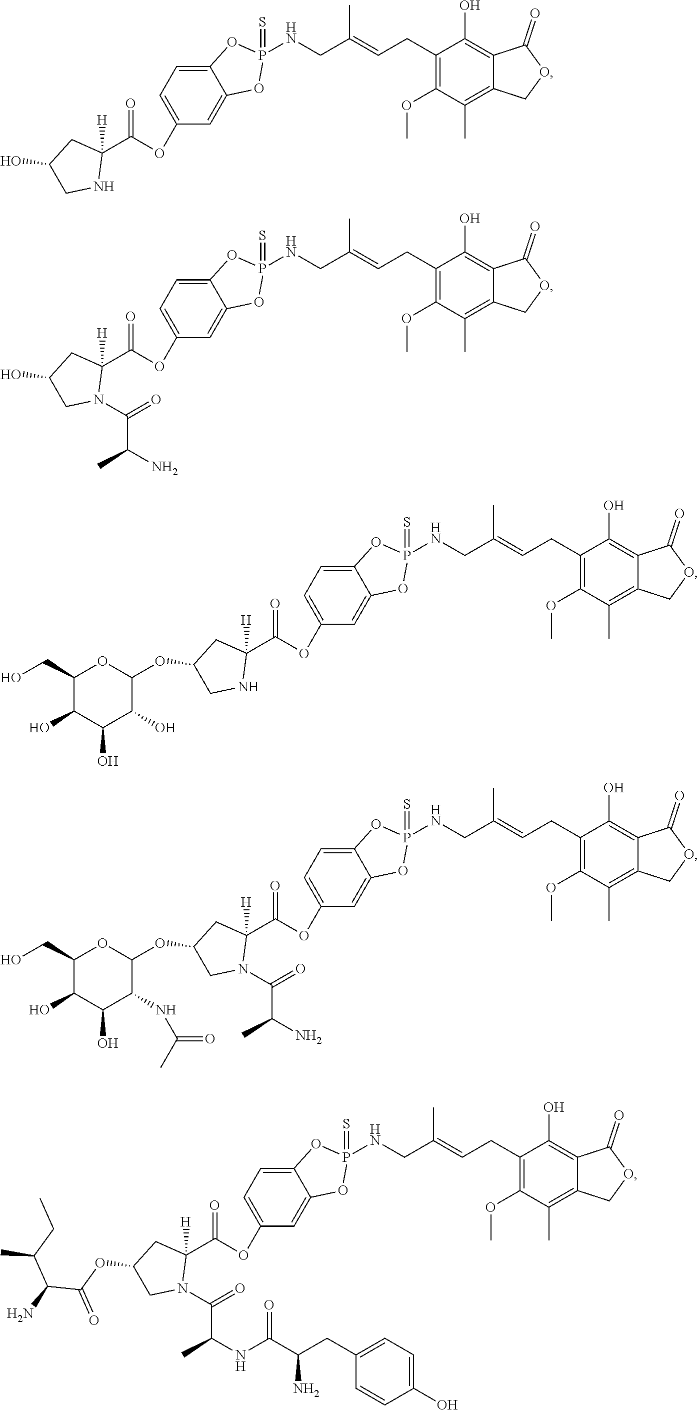

In another aspect, the disclosure provides a pharmaceutical formulation comprising a CSII compound as described herein, and a pharmaceutically acceptable carrier. In some embodiments, he pharmaceutical formulation, further comprising an immunomodulatory or anti-proliferative compound different than the CSII compound.

The disclosure also provides a method of treating a proliferative disorder in a patient, comprising administering to the patient an amount of a formulation as described herein effective to reduce or inhibit a symptom of the proliferative disorder. In some embodiments, the method comprising administering to the patient an amount of a CSII compound as described herein, in a pharmaceutically acceptable carrier, effective to reduce or inhibit at least one symptom of the proliferative disorder.

In yet another aspect, the disclosure provides a method of synthesizing a CSII compound as described herein, comprising carrying out the protocol of scheme 1 as set forth in FIG. 2A. The disclosure also provides a method of synthesizing a CSII compound as described herein, comprising carrying out the protocol of scheme las set forth in FIG. 2B.

In another aspect, the disclosure provides a method of synthesizing 1-[(2R,3R,4S,5R)-5-[[6-[[(2S)-2,6-diaminohexanoyl]-amino]-2-oxo-4H-1,3,2benzodioxaphosphinin-2-yl]oxymethyl]-3,4-dihydroxyetrahydrofuran-2-yl]-1,2,4-triazole-3-carboxamide, comprising carrying out the protocol set forth in EXAMPLE 1 and FIG. 2A.

In yet another aspect, the disclosure provides a method of synthesizing 1-[(2R,3R,4S,5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide, comprising carrying out the protocol set forth in EXAMPLE 2 and FIG. 2B.

The disclosure also provides a method of modulating the activity of an immune cell, comprising contacting the cell with a CSII as described herein, effective to reduce or inhibiting the activity of IMPDH.

### BRIEF DESCRIPTION OF THE DRAWING

The foregoing and other objects of the present disclosure, the various features thereof, as well as the disclosure itself may be more fully understood from the following description, when read together with the accompanying drawings in which:

FIG. 1A is a schematic representation of one nonlimiting general scheme for the preparation of CSII compounds of the disclosure;

FIG. 1B is a schematic representation of another nonlimiting general scheme for the preparation of CSII compounds of the disclosure;

FIG. 2A is a schematic representation of the synthesis of -[(2R,3R,4S,5R)-5-[[6-[[(2S)— 2,6-diaminohexanoyl]amino]-2-oxo-4H-1,3,2benzodioxaphosphinin-2-yl]oxymethyl]-2,6-diaminohexanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide using the protocol of Scheme 1; and

FIG. 2B is a schematic representation of the synthesis of [(2R,3R,4S,5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide was using the protocol of Scheme 2.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of +20% or +10%, including+5%, +1%, and +0.1% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with an infection an effective amount of an anti-infective formulation of the disclosure for conditions related to infections.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, but are not limited to, livestock and pets, such as ovine, bovine, porcine, canine, feline and marine mammals.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the CSII compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition”. “pharmaceutical composition”, or “formulation” refers to a CSII or a salt thereof, according to the disclosure in a pharmaceutically acceptable carrier.

An “oral dosage form” includes a unit dosage form prescribed or intended for oral administration.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. In an embodiment, C₁-C₆ alkyl groups are provided herein. Nonlimiting examples include methyl, ethyl, propyl, isopropyl, butyl, isobutyl, tert butyl, pentyl, neopentyl, and hexyl. Other nonlimiting examples Of C₁-C₆ alkyl include ethyl, methyl, isopropyl, isobutyl, n-pentyl, and n-hexyl.

The term “alkenyl” employed alone or in combination with other terms, refers to a straight-chain or branched hydrocarbon group corresponding to an alkyl group having one or more double carbon-carbon bonds, An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. For example, the alkenyl moiety contains 2 to 6, 2 to 4, or 2 to 3 carbon atoms. Exemplary alkenyl groups include, but are not limited tot ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to in carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “allyl” employed alone or in combination with other terms, refers to an sp³ carbon atom adjacent to an alkenyl group as defined above.

As used herein, the term “halo” or “halogen” alone or as part of another substituent means, unless otherwise stated, a fluorine, chlorine, bromine, or iodine atom.

As used herein, the term “aromatic” refers to a carbocycle or heterocycle with one or more polyunsaturated rings and having aromatic character, i.e., having (4n+2) delocalized π elections where n is an integer).

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. Aryl groups may have 6 carbon atoms, six to ten carbon atoms, or six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]-triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl.

It is to be understood that if an aryl and heteroaryl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, and the term “thienyl” means 2- or 3-thioenyl.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Cell Selective IMPDH Inhibitors

The present disclosure provides inosine-5′-monophosphate dehvdrogenase (IMPDH)-inhibiting small molecule compounds that are proliferative disease-modifying, immune modulatory, and immune cell type-selective (CS IMPDH inhibitors or “CSIIs”).

IMPDH is a purine biosynthetic enzyme which is highly conserved across all domains of life. As the de novo purine synthesis pathway is responsible for producing the bulk of guanine used for new RNA and DNA synthesis, the proper functioning of IMPDH is significant for the health of all rapidly proliferating biology, even viruses, By comparison, cells that are not actively dividing, such as most adult mammalian somatic cells, have relatively little demand for new nucleobases as they are less adversely affected by IMPDH inhibition. This difference in IMPDH dependency between slow and rapidly dividing cells provides a useful therapeutic index.

The CSIIs of the present disclosure contains a fully active IMPDH inhibitor moiety and thus do not require a conversion step to become therapeutic. Cleavage of the selectivity moiety and release of the active inhibitor occurs only when disease factors are present, thus sparing unaffected cells. Representative, nonlimiting disease factors include small molecules (chemical compounds) or macromolecules that circulate in plasma of a subject with active diseases (such as flare), but not in healthy subjects or subjects that have their diseases in remission.

The CSIIs according to the disclosure are optimized to achieve immune cell-selective accumulation, Exemplary immune cell types to which CSIIs are targeted include, but are not limited to, those in Table 1 below.

TABLE 1

| | Immune Phenotype |
|---|---|
| Immune Cells Population | |
| B Cell | CD19⁺ |
| “Helper” T | CD4⁺CD8⁻ |
| cells | |
| “Effector” T | CD8⁺CD4⁻ |
| cells | |
| Natural Killer (NK) | CD16⁺CD56⁺ |
| Cells | |
| Mast Cells | CD23⁺CD117⁺ |
| Neutrophils | CD10⁺ CD13⁺ CD15⁺ CD33⁺ CD141⁺ |
| | CD178⁺ |
| Basophils | CD9⁺ CD23⁺ CD24⁺ CD32⁺ CD43⁺ CD69⁺ |
| Eosiniphils | CD9⁺ CD23⁺ CD24⁺ CD32⁺ CD43⁺ CD49d⁺ |
| Monocytes | CD2⁺ CD11b⁺ CD14⁺ CD31⁺ CD62L⁺ |
| | CD115⁺ CD192⁺ |
| Macrophages | CD14⁺ CD16⁺ CD64⁺ CD68⁺ CD71⁺ CCR5⁺ |
| B Cell Subset | |
| Transitional | CD20⁺ CD27⁻ CD38^{hi} IgM⁺ CD24^{hi} BR3⁺ |
| Follicular | IgM^{lo} CD23⁺ CD93⁻ CD19⁺ CD20⁺ CD21⁺ |
| | CD22⁺ |
| Marginal zone | IgM^{hi} IgD^{lo} CD1c⁺ CD24⁺ CD19⁺ CD20⁺ |
| | CD21⁺ |
| Germinal center | CD20⁺ CD38⁺ BR3⁺ IgD⁻ |
| Plasma cells | CD20⁻ CD38^{hi} CD27^{hi} CD138⁺ TACI⁺ and/or |
| | BCMA⁺ CD126⁺ CD319⁺ CD78⁺ |
| Memory B Cell | CD20⁺ CD38⁻ CD27⁺ CD80⁺ CD84⁺ CD86⁺ |
| T Cell Subset | |
| CD8 Memory T cell | CD3⁺ CD8⁺ CD45RO⁺ |
| CD8 cytotoxic T | CD3⁺ CD8⁺ Granzyme A/B⁺ |
| cell | |
| Treg | CD25⁺ CD39⁺ CD73⁺ CD103 |
| CD4 memory T Cell | CD3⁺ CD4⁺ CD25⁺ CD45RO⁺ CD62L⁺ |
| Th1 | CD178⁺ CD183⁺ CD195⁺ CD212⁺ CD218α⁺ |
| Th2 | CD183⁺ CD193⁺ CD194⁺ CD198⁺ CD294⁺ |
| Th9 | CD196⁺ IL-9⁺ |
| Th17 | CD45RO⁺ CD126⁺ CD161⁺ CD194⁺ CD196⁺ |
| | IL-13α1⁺ IL-21R⁺ |
| Th22 | CCR10⁺ CD140a⁺ CD140b⁺ CD194⁺ CD196⁺ |
| Tfh | CD185⁺ CD279⁺ |
| Macrophage Subset | |
| M1 | CD68⁺ CD86⁺ CD282⁺ CD284⁺ |
| M2 | CD163⁺ CD200R⁺ CD206⁺ |
| TAM | CD81⁺ CD106⁺ Dectin-1⁺ VEGF⁺ |
| CD169⁺ macrophage | CD106⁺ CD169⁺ CD206⁺ |
| TCR⁺ macrophage | CD3⁺ ZAP70⁺ TCRα/β⁺ |

Cell type specificity can be conferred in a variety of ways. Differential permeability, differential activation of a prodrug, differential degradation, and differential enzymatic activation, and/or differential expulsion from different cell types are nonlimiting representative ways in which a drug can be activated preferentially in one cell type compared to others. For example, CSIIs according to the disclosure may be optimized to achieve immune cell-selective accumulation based on interaction with enzymes that are highly expressed in the particular immune cell.

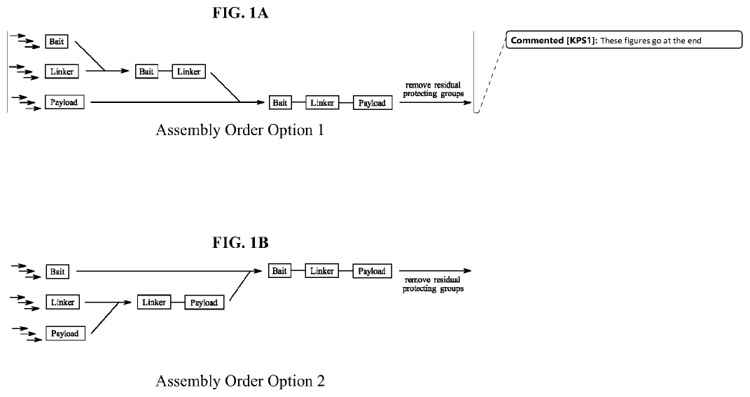

The CSIIs of the present disclosure achieve their uniqueness from their structure. As shown in FIG. 1, they consist three parts: (1) a “bait” or selective moiety, which is a substrate that can be cleaved by an enzyme that is highly expressed in the target cell type; (2) a “payload” moiety, which is the active inhibitor or modulator of IMPDH and is an antiproliferative agent; and (3) a “linker” moiety, which is unstable on its own but can be stabilized by contact with the bait and is placed between the bait and the payload.
BAIT----LINKER----PAYLOAD

This strategy enables only the target cell population to remove the bait group, allowing the linker to hydrolyze automatically thereby releasing the payload.

CSII compounds according to the disclosure fall into the generic structures of either Formula I or Formula 2 as set forth below.

A compound of Formula I comprises

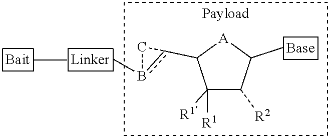

or a pharmaceutically acceptable salt thereof, wherein:
---- is an optional bond;
A is selected from the group consisting of CH₂, NH, O, and S;

B is selected from the group consisting of C, CH, CH₂, N, NH, O, and S;

C is selected from the group consisting of CH₂, NH, O, and S;

R¹ is selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{1′} is selected from the group consisting; of OH, SH, NH₂, N₃, and halo;
R² is selected from the group consisting of OH, SH, N-b, N₃, and halo;
Base is selected from the group consisting of

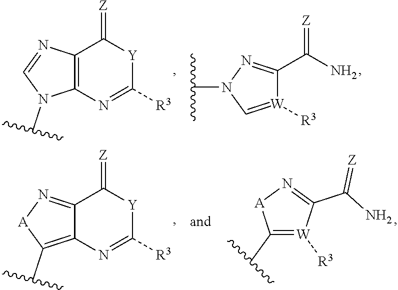

Z is selected from the group consisting of O, NH, and S;
Y is selected from the group consisting of O, NH, and S;
W is selected from the group consisting of CH, CH₂, NH, and N;

R³ is selected from the group consisting of OH, SH, NH₂, N₃, and halo;
Linker is selected from the group consisting of

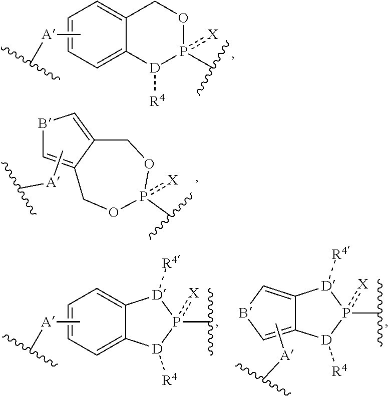

wherein:
A′ is independently, at each occurrence, selected from the group consisting of CH₂, NH, O, S, CO₃, CO₂N, CO₂S, and CO₂N;
B′ is independently, at each occurrence, selected from the group consisting of CH₂, NH, O, and S;
D and D′ are each independently selected from the group consisting of O, N, NH, and S;
X is selected from the group consisting; of O, NH—, and S;
R⁴ and R^{4′} are each independently selected from the group consisting of H, C₁₋₄ alkyl, C₂₋₄alkenyl, C₂₋₄ vinyl, and C₃₋₄ allyl, all of which are optionally substituted with one, two, three, or four halo;
Bait is an oligomer comprising 1-5 units, which are independently, at each occurrence, selected from the group consisting of

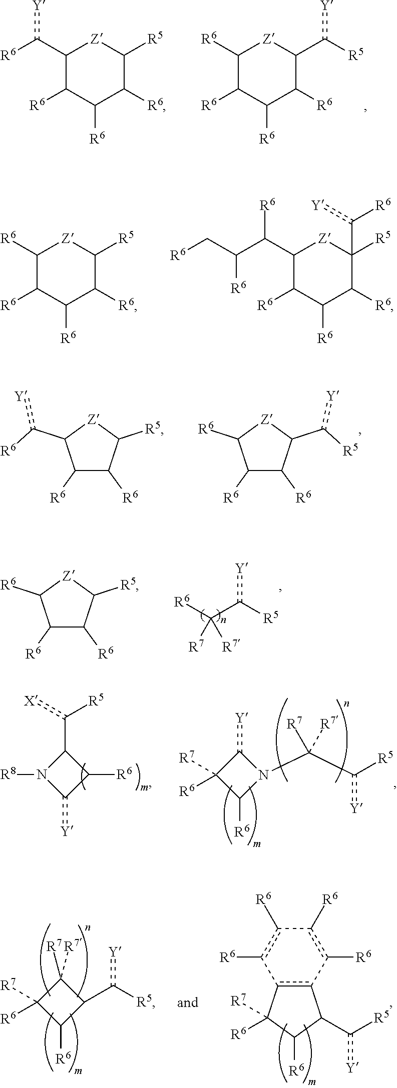

Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and a bond to A′;
R⁶ is independently, at each occurrence, selected from the group consisting of a bond to R⁵, H, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₁₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R^{7′} are each independently, at each occurrence, selected from the group consisting of H, NH₂, OH, C₆₋₁₀ aryl, 5-10 membered heteroaryl, C₁₋₄ alkyl, —(CH₂)₁₋₃C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1,2, or 3.

In one example of a compound of Formula T,

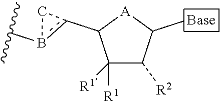

### is the Payload.

A compound of Formula I may be a compound of Formula Ia:

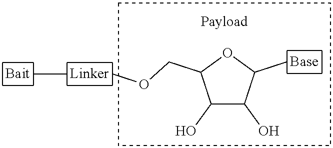

or a pharmaceutically acceptable salt thereof.

Alternatively, a compound of Formula I is a compound of Formula Ib:

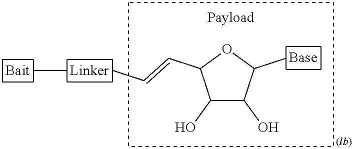

or a pharmaceutically acceptable salt thereof.

In the Formula I, Formula Ia, and Formula Ib compounds, the Base may comprise:

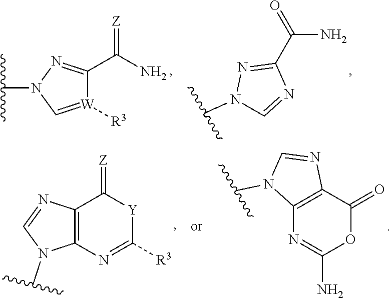

The Bait in these compounds may be a linear or branched oligomer, and there may be multiple Linkers and multiple baits.

The Linker in these compounds may comprises

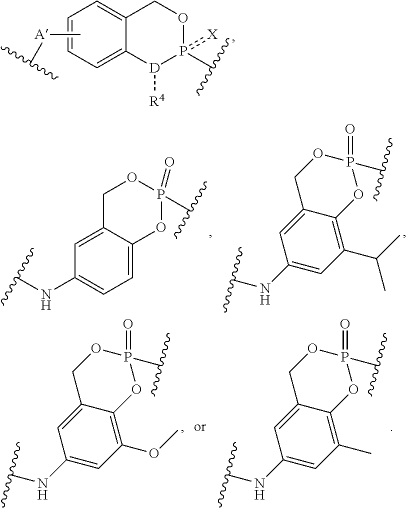

In specific nonlimiting examples, the compound of Formula I comprises:

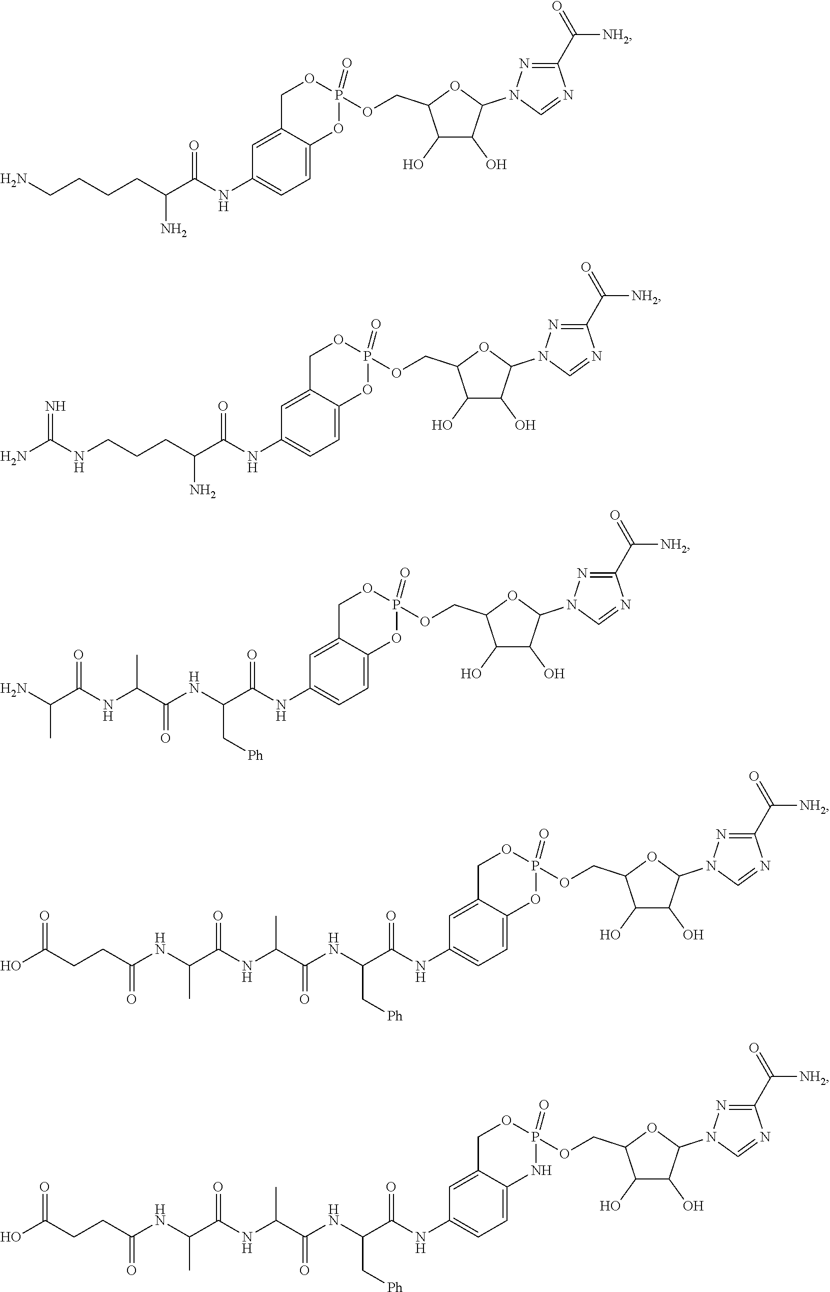 [SMILES: NCCCCC(N)C(=O)Nc1ccc2c(c1)COP(=O)(OCC1OC(n3cnc(C(N)=O)n3)C(O)C1O)O2 ; N=C(N)NCCCC(N)C(=O)Nc1ccc2c(c1)COP(=O)(OCC1OC(n3cnc(C(N)=O)n3)C(O)C1O)O2 ; CC(N)C(=O)NC(C)C(=O)NC(Cc1ccccc1)C(=O)Nc1ccc2c(c1)COP(=O)(OCC1OC(n3cnc(C(N)=O)n3)C(O)C1O)O2 ; CC(NC(=O)CCC(=O)O)C(=O)NC(C)C(=O)NC(Cc1ccccc1)C(=O)Nc1ccc2c(c1)COP(=O)(OCC1OC(n3cnc(C(N)=O)n3)C(O)C1O)O2 ; CC(NC(=O)CCC(=O)O)C(=O)NC(C)C(=O)NC(Cc1ccccc1)C(=O)Nc1ccc2c(c1)COP(=O)(OCC1OC(n3cnc(C(N)=O)n3)C(O)C1O)N2]

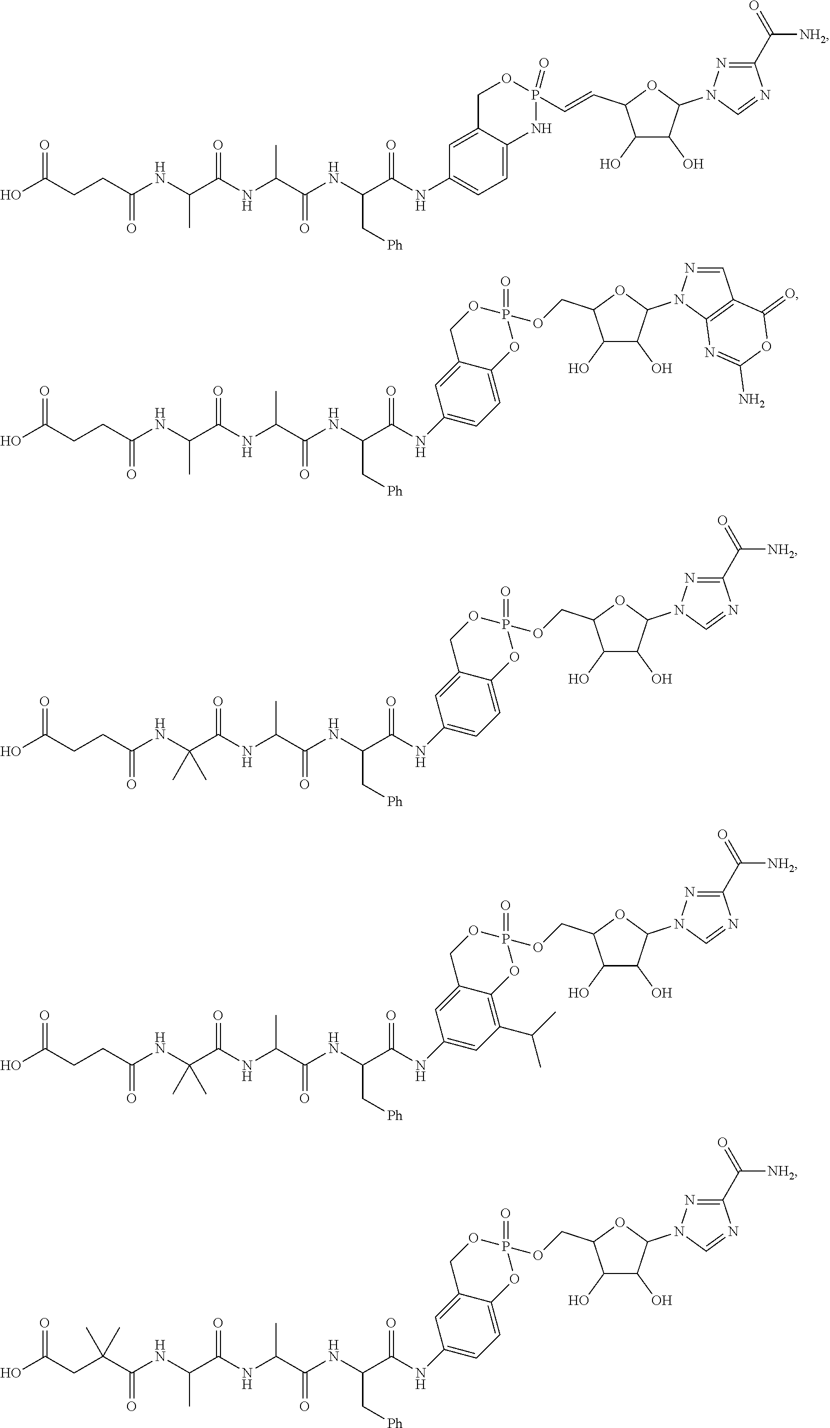

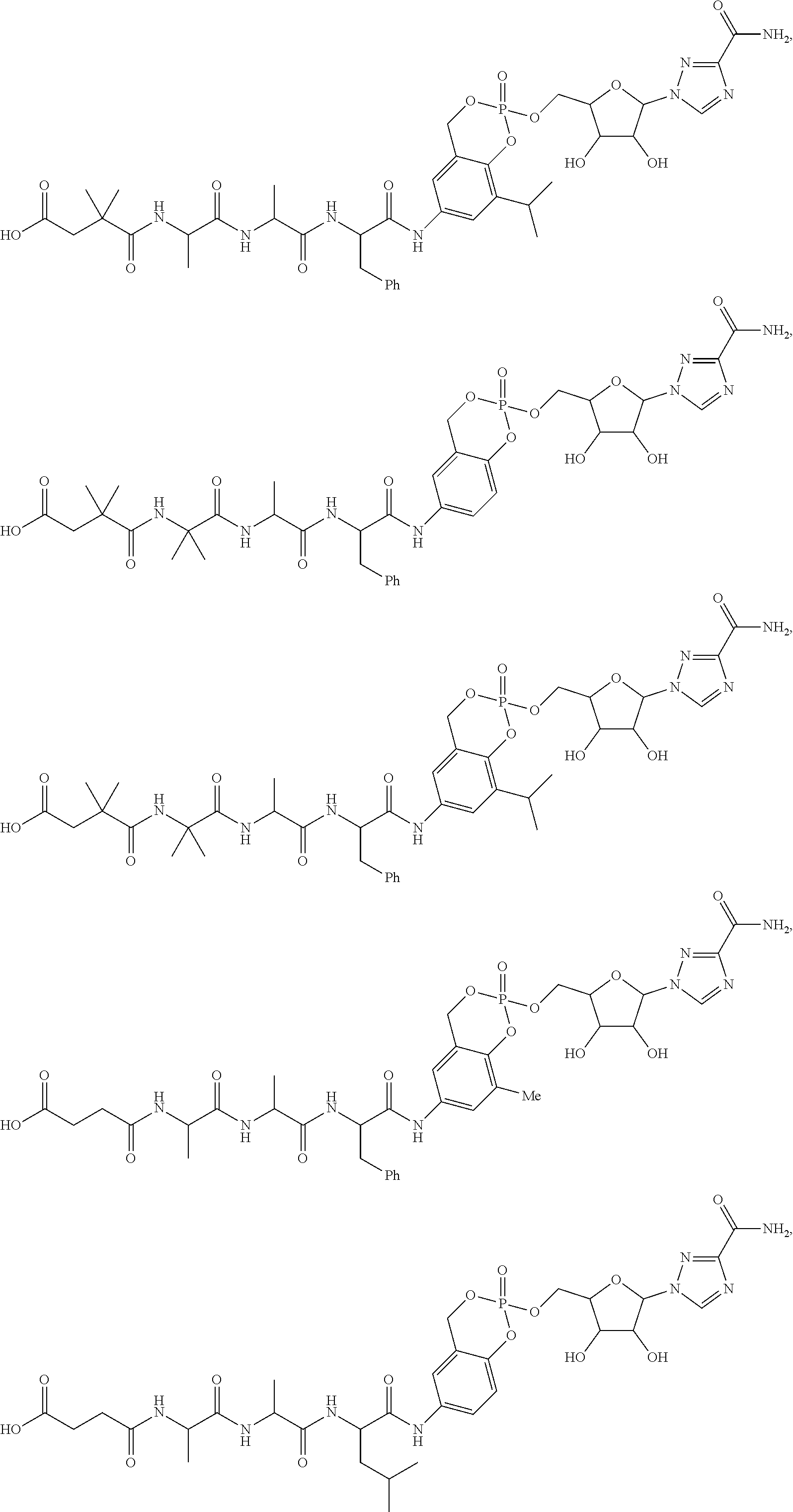

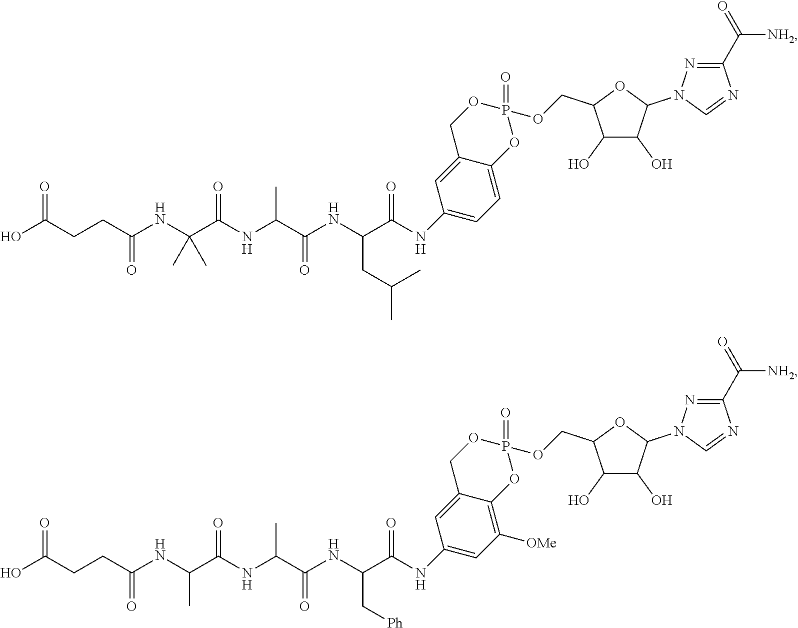 [SMILES: CC(C)CC(NC(=O)C(C)NC(=O)C(C)(C)NC(=O)CCC(=O)O)C(=O)Nc1ccc2c(c1)COP(=O)(OCC1OC(n3cnc(C(N)=O)n3)C(O)C1O)O2 ; COc1cc(NC(=O)C(Cc2ccccc2)NC(=O)C(C)NC(=O)C(C)NC(=O)CCC(=O)O)cc2c1OP(=O)(OCC1OC(n3cnc(C(N)=O)n3)C(O)C1O)OC2]

or a pharmaceutically acceptable salt thereof.

The compound of Formula I alternatively comprises a compound of Formula Ic:

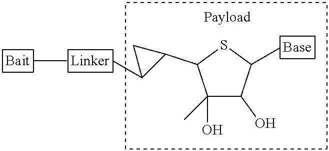

The Base in this compound may comprises

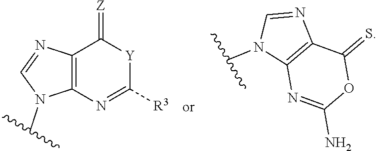

The Linker in this compound may comprise

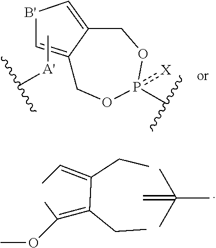

In specific nonlimiting examples, the compound of Formula Ic comprises:

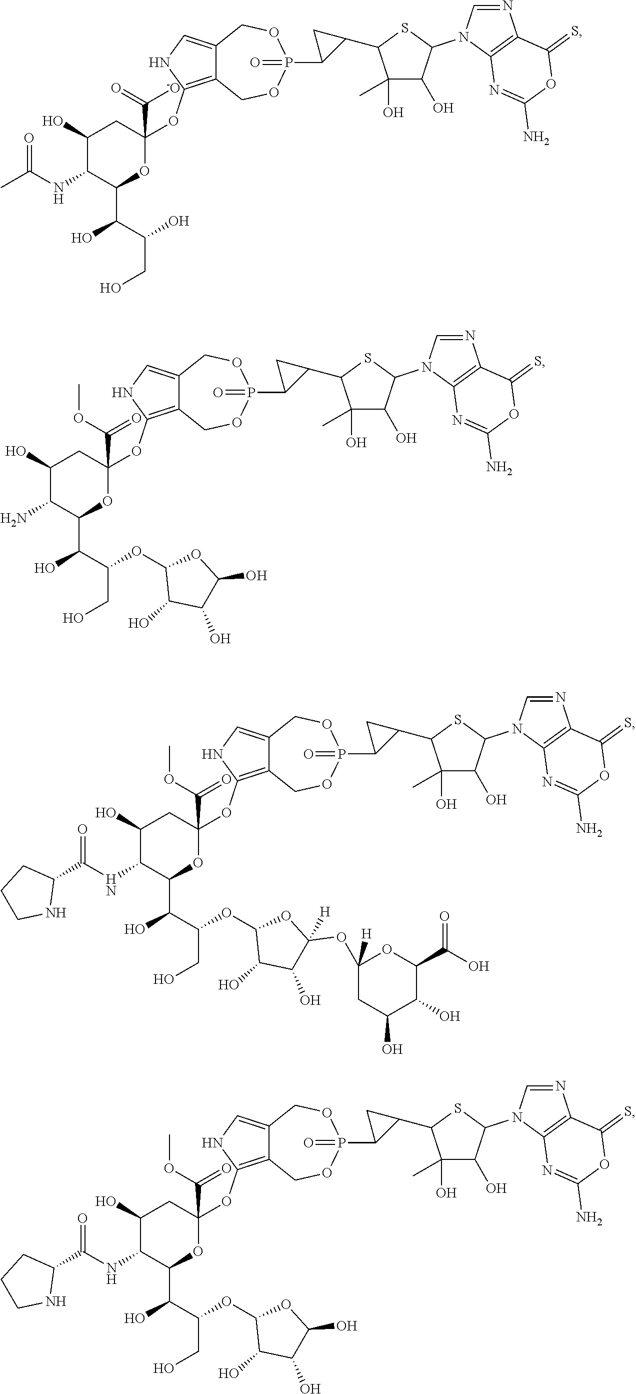

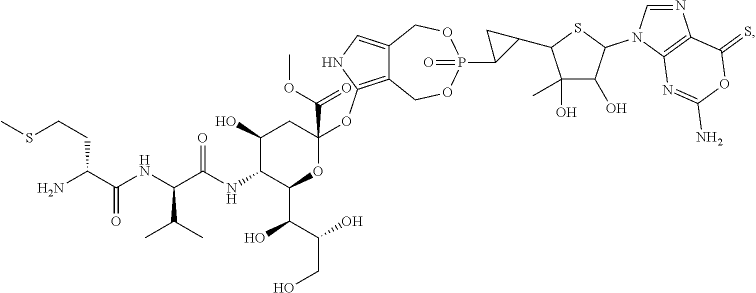

or a pharmaceutically acceptable salt thereof.

A CSII compound of Formula II comprises

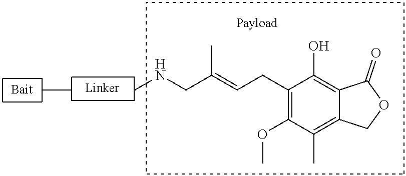

or a pharmaceutically acceptable salt thereof, wherein:
A is selected from the group consisting of CH₂, NH, O, and S;
W is independently, at each occurrence, selected from the group consisting of CH, C₂, NH, and N;
r is 1, 2, or 3;
Linker is selected from the group consisting of

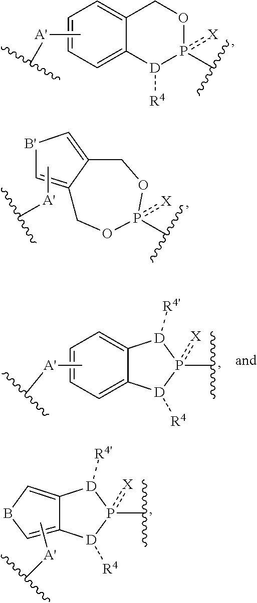

wherein:
A′ is independently, at each occurrence, selected from the group consisting of CH₂, NH, O, S, CO₃, CO₂N, CO₂S, and CO₂N;
B′ is independently, at each occurrence, selected from the group consisting of CH₂, NH, O, and S;
D and D′ are each independently selected from the group consisting of O, NH, and S;
X is selected from the group consisting of O, NH, and S; and
R⁴ and R^{4′} are each independently selected from the group consisting of H, C₁₋₄ alkyl, C₂₋₄ alkenyl, C₂₋₄ vinyl, and C₃₋₄ allyl, all of which are optionally substituted with one, two, three, or four halo;

Bait is an oligomer comprising 1-5 units, which are, independently at each occurrence selected from the group consisting of

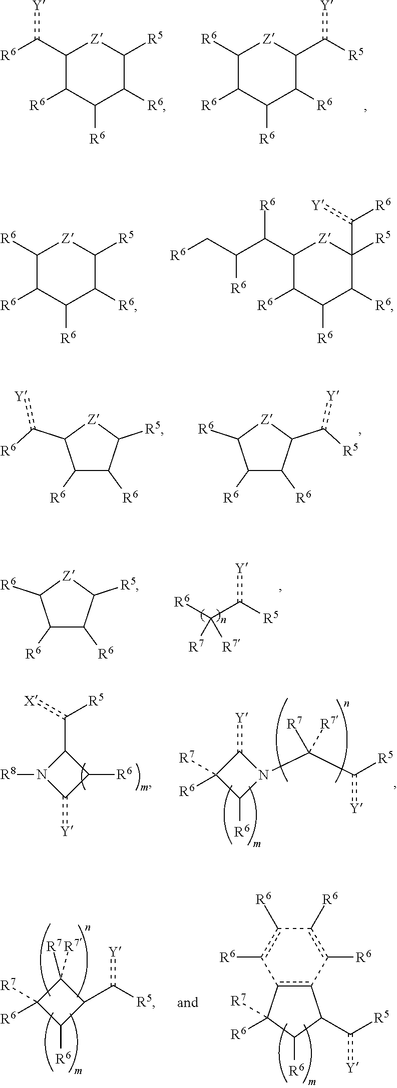

wherein:
Z is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and a bond to A′;
R⁶ is independently, at each occurrence, selected from the group consisting of a bond to R⁵, H, ═O, ═NH, S, —R^{A}—(C═R^{B})—R^{C}—, —R^{A}(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀-aryl, and —(CH₂)₁₋₃-5-10 membered heteroaryl; wherein aryl, heteroaryl, and alkyl are optionally substituted with one, two, or three substituents selected from the group consisting of halo, O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, —R^{A}(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1, 2, or 3.

One representative, nonlimiting Payload of a compound of Formula II comprises

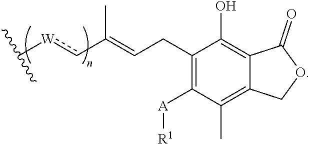

A compound of Formula II can be a compound of Formula IIa:

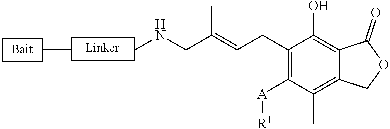

or a pharmaceutically acceptable salt thereof.

In the compounds of Formula II or IIa, the Linker can comprise

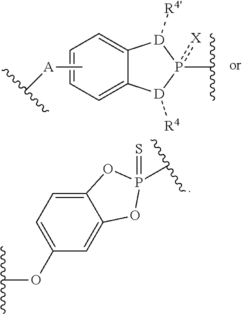

In specific nonlimiting examples, the CSII compound of Formula II comprises:

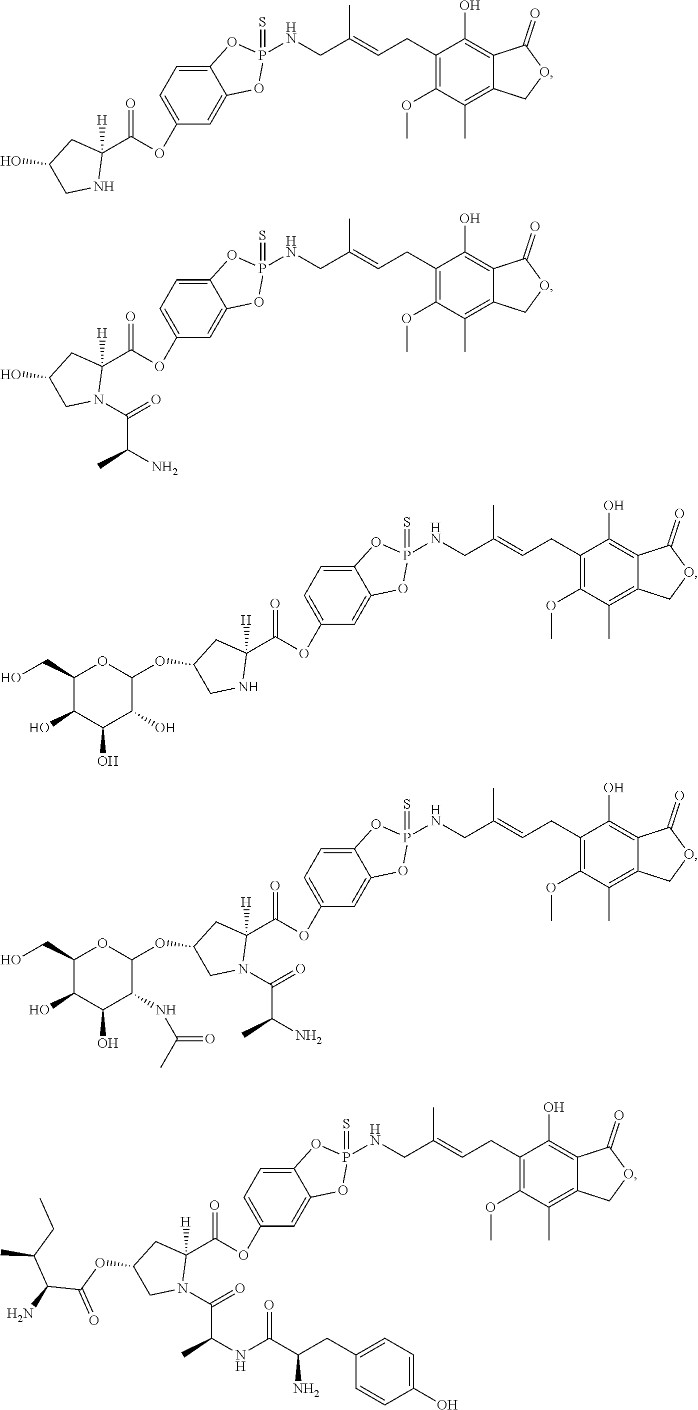

or a pharmaceutically acceptable salt thereof.

The CSII compounds described herein can be asymmetric (e.g., having one or more stereocenters). All stereoisomers of such compounds, such as enantiomers and diastereomers, are intended unless otherwise indicated.

CSII compounds of the present disclosure that contain asymmetrically substituted carbon atoms can be isolated in optically active or racemic forms. Methods on how to prepare optically active forms from optically inactive starting materials are known in the art, such as by resolution of racemic mixtures or by stereoselective synthesis.

Many geometric isomers of olefins, C═N double bonds and the like can also be present in the compounds described herein, and all such stable isomers are contemplated in the present disclosure. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as +/− camphorsulfonic acid. Other resolving agents suitable for fractional crystallization methods include stereoisomerically pure forms of +/− methylbenzylamine (e.g., S and R forms, or diastereomerically pure forms), 2-phenylglycinol, norephedrine, ephedrine, N-methylephedrine, cyclohex ylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

The CSII compounds of the present disclosure can have the (R)-configuration or the (S)-configuration. In compounds with more than one chiral center, each of the chiral centers in the compound may be independently (R) or (S), unless otherwise indicated.

CSII compounds according to the present disclosure also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge, Example prototropic tautomers include ketone-enol pairs, amide-imidic acid pairs, lactam-lactim pairs, enamine-imine pairs, and annular forms where a proton can occupy two or more positions of a heterocyclic system, e.g., 1H— and 3H-imidazole, 1H—, 2H— and 4H-1,2,4-triazole, 1H— and 2H— isoindole and 1H— and 2H-pyrazole. Tautomeric forms can be in equilibrium or sterically locked into one form by appropriate substitution.

Exemplary IMPDH inhibitor according to the disclosure includes as a Payload ribavirin-monophosphate (RMP), the active form of ribavirin or mizoribine-monophosphate (MMP), the active form of mizoribine. Such nucleotide analogs are amenable to a variety of cell-selection approaches.

### Syntheses

Synthesis of a CSII compound according to the disclosure comprises: preparation of the Bait, Linker, and Payload, separately, with any free aliphatic amitnes or protic functionality with a pKa<20 in DMSO protected; assembly in either one of two orders as modules per the depiction in FIGS. 1A and 1B; and then removal of any residual protecting groups to yield the final CSII compound.

Baits are individually synthesized from amino acid or sugar components via standard methods for oligopeptide synthesis (e.g., Jaradat (2017) Amino Acids. 50 (1): 39-68) and/or oligosaccharide synthesis (e.g., Levy et al. The Organic Chemistry of Sugar; Taylor & Francis: 2006.) depending on the composition of a given Bait. For a Bait to be ready to be attached to a linker, a single R⁵ position that is the point of attachment to the linker is left un-protected, while all other aliphatic amines and protic functionality with a pKa<20 in DMSO are protected via standard means (e.g., Wuts and Green; Greene 'S Protective Groups in Organic Synthesis, Fourth Edition; John Wiley & Sons, Inc.: 2007).

The syntheses of two representative CSIIs are shown in EXAMPLES 1 and 2.

### Disorders to be Treated

The CSIIs according to the disclosure are useful for treating proliferative disorders impacting immune cells. For example, certain lymphomas affecting T cells (T-cell lymphomas including Extranodal T cell lymphoma, Cutaneous T-cell lymphoma, Anaplastic large cell lymphoma and Angioimmunoblastic T-cell lymphoma), B cells (B-cell lymphomas including Hodgkin's lymphoma, nodular lymphocyte predominant Hodgkin's lymphoma, Diffuse large B-cell lymphoma (DLBCL), Follicular lymphoma, Marginal zone B-cell lymphoma (MZL), Mucosa-Associated Lymphatic Tissue lymphoma (MALT), Small lymphocytic lymphoma (also known as chronic lymphocytic leukemia, CLL), Mantle cell lymphoma (MCL), Burkitt's lymphoma, Lymphoplasmacytic lymphoma, which may manifest as Waldenström's macroglobulinemia, Nodal marginal zone B cell lymphoma (NMZL), Splenic marginal zone lymphoma (SMZL), Intravascular large B-cell lymphoma, Primary effusion lymphoma, Lymphomatoid granulomatosis, Primary central nervous system lymphoma, ALK-positive large B-cell lymphoma, Plasmablastic lymphoma, Large B-cell lymphoma arising in HHV8-associated multicentric Castleman's disease) can be treated using these molecules. Immune cell leukemia including Acute lymphoblastic leukemia (ALL), Chronic lymphocytic leukemia (CLL), Acute myelogenous leukemia (AML), Chronic myelogenous leukemia (CML), Hairy cell leukemia (HCL), T-cell prolymphocytic leukemia (T-PLL), Large granular lymphocytic leukemia, Adult T-cell leukemia, Clonal eosinophilias (also called clonal hypereosinophilia) can also be treated using these molecules.

Other proliferative disorders that can be treated are inflammatory diseases such as atherosclerosis (AS), where macrophages play a central role, Asthma (involving T cells, nacrophages, neutrophils, eosinophils) can also be treated.

Particular autoimmune diseases are also treatable with these inhibitors. For example, rheumatoid arthritis (RA), inflammatory bowel diseases (IBD), celiac disease, diabetes mellitus type 1, Graves' disease, multiple sclerosis (MS), systemic lupus erythematosus (SLE), Sarcoidosis, pemphigus vulgaris (mostly B cell driven), myasthenia gravis (mostly B cell driven), and psoriasis can be treated,

### Pharmaceutical Formulations and Treatment

The CSIIs according to the disclosure are useful in in pharmaceutical formulations that treat proliferative disorders. These pharmaceutical formulations include a therapeutically effective amount of a CSII which has anti-proliferative properties a pharmaceutically acceptable carrier.

The term “pharmaceutically acceptable carrier” is to be understood herein as referring to any substance that may, medically, be acceptably administered to a patient, together with ta derivative according to the disclosure, and which does not undesirably affect the pharmacological and synergistic activity of the inhibitor. A “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington, The Science and Practice of Pharmacy (9th Ed. 1995).

The CSIs of the present disclosure can also be in the form of conventional, non-toxic salts which can be prepared, for example, from non-toxic inorganic or organic acids. The pharmaceutically acceptable CSII salts of the present disclosure can be synthesized from the parent CSII which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as, but no limited to, ether, ethyl acetate, ethanol, isopropanol, or acetonitrile are useful. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences. 17th ed., Mack Publishing Company, Easton, Pa., 1985, p. 1418 and Journal of Pharmaceutical Science, 66:2 (1977).

For use in medicine, the salts of the anti-proliferative CSIIs are pharmaceutically acceptable salts. Other salts may, however, be useful in the preparation of the CSIls or of their pharmaceutically acceptable salts according to the disclosure. Suitable pharmaceutical-salts of the CSIIs according to the present disclosure include acid addition salts which may, for example, be formed by mixing a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Additionally, where the anti-infective CSIIs in the formulation carries an acidic moiety, suitable pharmaceutically acceptable salts thereof may include alkali metal salts, e.g., sodium or potassium salts; alkaline earth metal salts, e.g. calcium or magnesium salts; and salts formed with suitable organic ligands, e.g., quaternary ammonium salts.

The pharmaceutical formulations may be prepared for injectable use, topical use, oral use, intranmuscular or intravenous injection, inhalation use, transdermal use, intradermal, transmembrane use, and the like. These formulations are in unit dosage forms such as tablets, pills, capsules, powders, granules, sterile parenteral solutions or suspensions, metered aerosol or liquid (nebulized) sprays, drops, ampoules, auto-injector devices or suppositories; for oral parenteral, intranasal, sublingual topical or rectal administration, or for administration by inhalation or insufflation. Alternatively, the formulations may be presented in a form suitable for one-weekly or once-monthly administration; for example, an insoluble salt of the derivative, such as decanoate salt, may be adapted to provide a depot preparation for intramuscular injection. An erodible polymer containing the inhibitor may also be envisaged.

For preparing solid compositions such as tablets, the CSII is mixed with a pharmaceutical carrier, e., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a CSII of the present disclosure, or a pharmaceutically acceptable salt thereof.

These formulations may be homogeneous, i.e., the derivative is dispersed evenly throughout the composition so that the composition may be readily subdivided into equally effective unit dosage forms such as tablets, pills and capsules. This solid formulation composition is then subdivided into unit dosage forms of the type described above containing from about 0.1 mg to about 500 ng of the CSII. Some useful unit dosage forms contain from mg to 100 mg, for example, about 1 mg, about 2 mug, about 5 mg, about 10 mg, about 25 mg, about 50 mug, or about 100 mg, of the CSII. The tablets or pills comprising the CSIls can be coated or otherwise compounded to provide a dosage form affording the advantage of prolonged action. The liquid forms in which the CSIIs of the present disclosure may be incorporated for administration orally or by injection include aqueous solutions, suitably flavored syrups, aqueous or oil suspensions, and flavored emulsions with edible oils as well as elixirs and similar pharmaceutical vehicles.

Injectable dosage forms may be sterilized in a pharmaceutically acceptable fashion, for example by steam sterilization of an aqueous solution scaled in a vial under an inert gas atmosphere at 120° C. for about 15 minutes to 20 minutes, or by sterile filtration of a solution through a 0.2 μM or smaller pore-size filter, optionally followed by a lyophilization step, or by irradiation of a composition containing an inhibitor of the present disclosure by means of emissions from a radionuclide source.

A therapeutically effective dosage of the formulation according to the disclosure depends on the disorder treated and may vary from patient to patient, and factors such as the age and physical size of the patient, the patient's genetics, and the diagnosed condition of the patient, and the route of delivery of the dosage form to the patient. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 0.1 mg/kg to about 1000 mg/kg, or from about 1 mg/kg to about 100 mg/kg inhibitor may be suitable. For example, for the treatment of proliferation disorders, a suitable dosage level is about 0.001 mg/kg to about 250 mg/kg per day. The formulation maybe administered on bolus and or a regimen of about 1 to 4 times per day.

Reference will now be made to specific examples illustrating the disclosure. It is to be understood that the examples are provided to illustrate exemplary embodiments and that no limitation to the scope of the disclosure is intended thereby.

### EXAMPLES

### Example 1

Synthesis of

1-[2R,3R,4S,5R)-5-[[6[[(2S)-2,6-diaminohexanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide Using Scheme 1

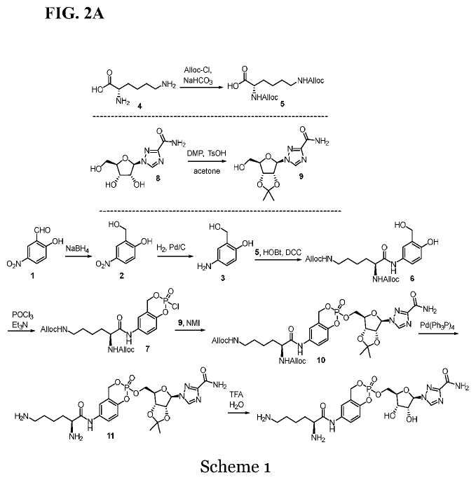

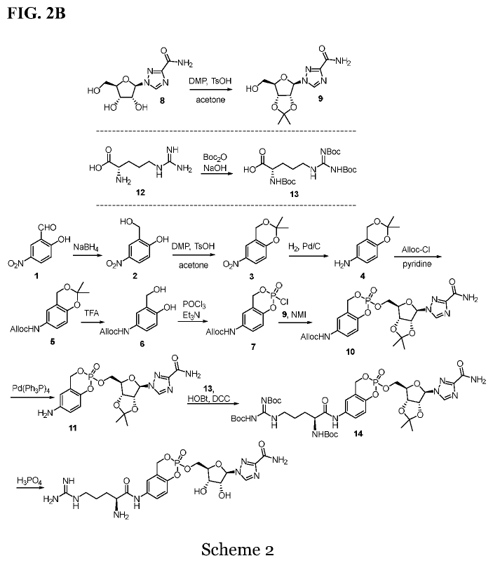

1-[(2R,3R,4S,5R)-5-[[6-[[(2S)-2,6-diaminohexanoyl]amino]-2-oxo-4H-1,3,2benzodioxaphosphinin-2-yl]oxymethyl]-3,4-dihydroxy-tetrahydrofuran-2-yl]-1,2,4-triazole-3-carboxamide was synthesized according to the protocol of scheme 1 (FIG. 2A). Briefly:

Step 1: (2R)-2,6-bis(allyloxycarbonylamino)hexanoic acid

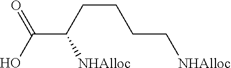

(2R)-2,6-diaminohexanoic acid (4.00 g, 27.36 mmol, 1 eq) was dissolved in H₂O (20.5 mL). The solution was cooled in an ice-bath and treated with NaOH (3.28 g, 82.08 mmol, 3 eq) and allyl carbonochloridate (6.60 g, 54.72 mmol, 5.79 mL, 2 eq). The mixture was stirred at 20° C. for 12 hr. The mixture was acidified to congo red with conc. hydrochloric acid, then extracted with ethyl acetate (200 mL*3), the organic layer was washed with saturated brine, dried over anhydrous magnesium sulfate, filtered, and the filtrate was concentrated. The residue was purified by column chromatography (SiO₂, Dichloromethane: Methanol=:100/1 to 20:1) to give (2R)-2,6-bis(allyloxycarbonylamino) hexanoic acid (7.2 g, 22.91 mmol, 83.72% yield) as a colorless oil. (MS: [M+1]315.1)

Step 2: 2-(hydroxymethyl)-4-nitro-phenol

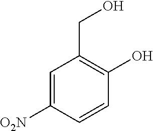 [SMILES: O=[N+]([O-])c1ccc(O)c(CO)c1]

To a solution of 2-hydroxy-5-nitro-benzaldehyde (10 g, 59.84 mmol, 1 eq) in EtOH (100 mL) was added NaBH₄ (4.53 g, 119.68 mmol, 2 eq) in portions. The mixture was stirred at 20° C. for 24 hr. To the reaction mixture was added 200 mL of 3M aq. HCl, and then was extracted with EA (400 mL). The organic layer was concentrated. The residue was purified by column chromatography (SiO₂, DCM: MeOH=20:1) to give 2-(hydroxymethyl)-4-nitro-phenol (7 g, 41.39 mmol, 69.17% yield) as yellow solid.

Step 3: 4-amino-2-(hydroxymethyl)phenol

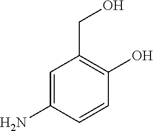

To a solution of 2-(hydroxymethyl)-4-nitro-phenol (4 g, 23.65 mmol, 1 eq) in MeOH (30 mL) was added Pd/C (0.4 g, 2.36 mmol, wt. 10%, 0.1 eq) under N₂ atmosphere. The suspension was degassed and purged with H₂ for 5 times. The mixture was stirred under H₂ (15 Psi) at 20° C. for 12 hr. The mixture was filtered off and the solvent was removed, 4-amino-2-(hydroxy-methyl)phenol (3.5 g, crude) was obtained as slightly purple solid.

Step 4: allyl N-[(5R)-5-(allyloxycarbonylamino)-6-[4-hydroxy-3-(hydroxymethyl)anilino]-6-oxo-hexyl]carbamate

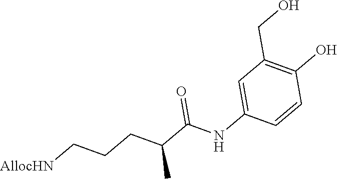

(2R)-2,6-bis(allyloxycarbonylamino)hexanoic acid (7.91 g, 25.15 mmol, 1 eq), 4-amino-2-(hydroxymethyl)phenol (3.5 g, 25.15 mmol, 1 eq) and HOBt (3.74 g, 27.67 mmol, 1.1 eq) were dissolved in dry DMF (10 mL), and then the mixture was cooled to 0° C., DCC (5.71 g, 27.67 mmol, 1.1 eq) was added and the mixture was stirred at 20° C. for 12 hr. The mixture was extracted with ethyl acetate (500 mL*3), the organic layer was washed with saturated brine, dried over anhydrous magnesium sulfate, filtered, and the filtrate was concentrated. The residue was purified by column chromatography (SiO₂, Ethyl acetate: Petroleum ether=100/1, 1/1) to give desired allyl N-[(5R)-5-(allyloxycarbonylamino)-6-[4-hydroxy-3-(hydroxy-methyl)anilino]-6-oxo-hexyl]carbamate (5.0 g, 11.48 mmol, 45.65% yield) as slightly purple oil. (MS: [M+1]436.1)

Step 5: allyl N-[(5S)-5-(allyloxycarbonylamino)-6-[(2-chloro-2-oxo-4H-1,3,2benzodioxa-phosphinin-6-yl)amino]-6-oxo-hexyl]carbamate

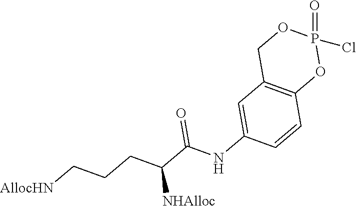

To a solution of allyl N-[(5S)-5-(allyloxycarbonylamino)-6-[4-lhydroxy-3-(hydroxymethyl)-anilino]-6-oxo-hexyl]carbamate (2 g, 4.59 mmol, 1 eq) in THE (30 mL) was added TEA (1.39 g, 13.78 mmol, 1.92 mL, 3 eq) in one portion, then POCl₃ (1.06 g, 6.89 mmol, 640.20 μL, 1.5 eq) was added dropwise at −40° C. and stirred at −40˜20° C. for 6 hrs. The reaction mixture was filtered and the solvent was removed under reduced pressure. The residue was used to next step without further purification. allyl N-[(5S)-5-(allyloxycarbonylamino)-6-[(2-chloro-2-oxo-4H-1,3,2benzodioxaphosphinin-6-yl)amino]-6-oxo-hexyl]carbamate (2.2 g, crude) was obtained as yellow oil.

Step 6: 1-[(3aR,4R,6R,6aR)-6-(hydroxymethyl)-2,2-dimethyl-3a,4,6,6a-tetrahydrofuro[3,4-d]-[1,3]dioxol-4-yl]-1,2,4-triazole-3-carboxamide

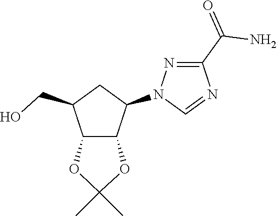 [SMILES: CC1(C)O[C@@H]2[C@@H](CO)C[C@@H](n3cnc(C(N)=O)n3)[C@@H]2O1]

To a solution of 1-[(2R,3R,4S,5R)-3,4-dihydroxy-5-(hydroxymethyl)tetra-hydrofuran-2-yl]-1,2,4-triazole-3-carboxamide (21 g, 85.99 mmol, 1 eq) in acetone (200 mL) was added 2,2-dimethoxypropane (17.00 g, 163.23 mmol, 20 mL, 1.90 eq) and TsOH.H₂O (2.8 g, 14.72 mmol, 0.17 eq) in one portion, then the mixture was stirred at 70° C. for 0.5 hr. The mixture was diluted with saturated NaHCO₃ aq. (15 mL), then it was evaporated to give a residue, which was purified by column chromatography (SiO₂, DCM/MeOH=50/1 to 15:1) to give 1-[(3aR,4R,6R,6aR)-6-(hydroxymethyl)-2,2-dimethyl-3a,4,6,6a-tetrahydrofuro[3,4-d][1,3]-dioxol-4-yl]-1,2,4-triazole-3-carboxamide (21 g, 73.87 mmol, 85.91% yield) as white solid. (MS: [M+1]285.1).

Step 7: allyl N-[(5S)-6-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzo-dioxaphosphinin-6-yl]amino]-5-(allyloxycarbonylamino)-6-oxo-hexyl]carbamate

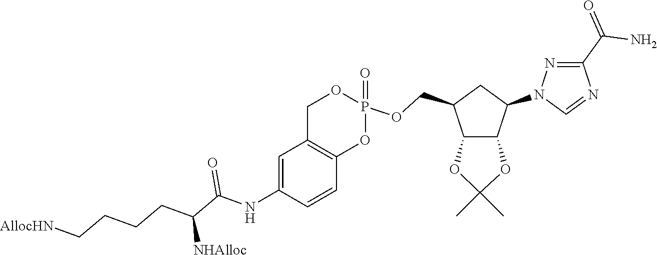

To a solution of 1-[(3aR,4R,6R,6aR)-6-(hydroxymethyl-2,2-dimethyl-3a,4,6,6a-tetrahydrofuro[3,4-d][1,3]dioxol-4-yl]-1,2,4-triazole-3-carboxamide (1.43 g, 5.04 mmol, 1.3 eq) in DCM (50 mL) was added allyl N-[(5S)-5-(allyloxycarbonylamino)-6-[(2-chloro-2-oxo-4H-1,3,2-benzodioxaphosphinin-6-yl)amino]-6-oxo-hexyl]carbamate (2 g, 3.88 mmol, 1 eq) in one portion, then N-methylimidazole (1.59 g, 19.38 mmol, 5 eq) was added dropwise at 20° C. and was stirred at 20° C. for 1 hr. The reaction mixture was evaporated to give the residue. The residue was purified by reversed-phase column (0.1% HCOOH in 0%˜70% water/CH₃CN) to give the desired peak, after lyophilization, got the crude product. allyl N-[(5S)-6-[[2-[[(3aR,4R-6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]amino]-5-(allyloxycarbonylamino)-6-oxo-hexyl]carbamate (1.5 g, crude) was obtained as yellow solid. (MS: [M+1]764.4).

Step 8: 1-[(3aR,4R-6R,6aR)-6-[[6-[[(2S)-2,6-diaminohexanoyl]amino]-2-oxo-4H-1,3,2benzo-dioxaphosphinin-2-yl]oxymethyl]-2,2-dimethyl-3a,4,6,6a-tetrahydrofuro[3,4-d][1,3]-dioxol-4-yl]-1,2,4-triazole-3-carboxamide

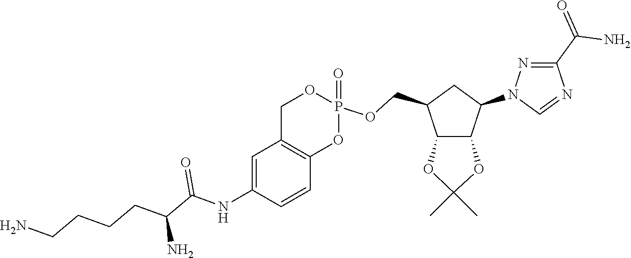 [SMILES: CC1(C)O[C@@H]2[C@@H](COP3(=O)OCc4cc(NC(=O)[C@@H](N)CCCCN)ccc4O3)C[C@@H](n3cnc(C(N)=O)n3)[C@@H]2O1]

To a solution of ally N-[(5S)-6-[[2-[[(3aR,4R,6R,6aR)-4-(3-carbamoyl-1,2,4-triazol-1-yl)-2,2-dimethyl-3a,4,6,6a-tetrahydrofuro[3,4-d][1],3]dioxol-6-yl]methoxy]-2-oxo-4H-1,3,2-benzodioxaphosphinin-6-yl]amino]-5-(allyloxycarbonylamino)-6-oxo-hexyl]carbamate (500 mg, 654.72 μmol, 1 eq) in DCM (20 mL) was added 1,3-dimethylhexahydropyrimidine-2,4,6-trione (408.91 mg, 2.62 mmol, 4 eq) and Pd(PPh₃)₄ (113.48 mg, 98.21 μmol, 0.15 eq) in one portion. The mixture was stirred at 20° C. for 1 hr. The reaction mixture was evaporated to give the residue. The residue was purified by reversed-phase column (0.1% HCOOH in 0%˜-70% water/C₃CN) to give the desired peak, after lyophilization, got crude 1-[(3aR,4R-6R-6aR)-6-[[6-[[(2S)-2,6-diaminohexanoyl]amino]-2-oxo-4H-1,3,2benzodioxa-phosphinin-2-yl]oxymethyl]-2,2-dimethyl-3a,4,6,6a-tetrahydrofuro[3,4-d][1,3]dioxol-4-yl]-1,2,4-triazole-3-carboxamide (200 mg, crude) as white solid. (MS: [M+1]596.3).

Step 9: 1-[(2R,3R,4S,5R)-5-[[6-[[(2S)-2,6-diaminohexanoyl]amino]-2-oxo-4H-1,3,2benzodioxa-phosphinin-2-yl]oxymethyl]-3,4-dihydroxy-tetrahydrofuran-2-yl]-1,2,4-triazole-3-carboxamide

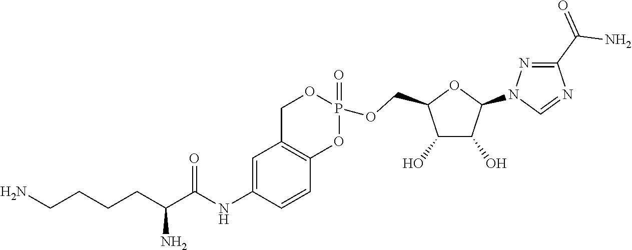 [SMILES: NCCCC[C@H](N)C(=O)Nc1ccc2c(c1)COP(=O)(OC[C@H]1O[C@@H](n3cnc(C(N)=O)n3)[C@H](O)[C@@H]1O)O2]

To a solution of 1-[(3aR,4R,6R-6aR)-6-[[6-[[(2S)-2,6-diaminohexanoyl]amino]-2-oxo-4H-1,3,2benzodioxaphosphinin-2-yl]oxymethyl]-2,2-dimethyl-3a,4,6,6a-tetrahydrofuro[3,4-d][1,3]dioxol-4-yl]-1,2,4-triazole-3-carboxamide (150 ng, 251.87 μmol, 1 eq) in H₂O (0.5 mL) was added TFA (2 mL) in one portion, then the mixture was stirred at 20° C. for 5 min. Water (5 mL) was added into the reaction mixture. The mixture was purified by reversed-phase column (0.1% HCOOH in 0%˜70% water/CH₃CN) to give the desired peak, after lyophilization, got 1-[(2R,3R,4S,5R)-5-[[6[[(2S)-2,6-diaminohexanoyl]amino]-2-oxo-4H-1,3,2benzodioxaphosphinin-2-yl]oxymethyl]-3,4-dihydroxy-tetrahydrofuran-2-yl]-1,2,4-trazole-3-carboxamide (22.5 mg, 40.51 μmol, 16.08% yield) as white solid. (MS: [M+1]556.3); ^{J}H NMR (MeOD, 400 MHz): δ(ppm) 8.58-8.54 (m, 1H), 8.34 (s, 1H—), 7.50-7.41 (m, 2H), 7.02-6.99 (m, 1H), 5.92-5.90 (m, 1H), 5.45-5.31 (m, 3H), 4.51-4.42 (m, 5H), 3.95-3.94 (m, 1H), 2.99-2.97 (m, 3H). 2.05-1.95 (m, 21H), 1.76-1.73 (m, 3H), 1.59-1.56 (m, 3H); ³¹p NMR (MeOD, 162 MHz): δ(ppm)−9.43, −9.63.

### Example 2

Synthesis of

1-[(2R,3R,4S 5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide Using Scheme 2

[(2R,3R,4S,5R)-5-[[6[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide was prepared according to the protocol of scheme 2 (FIG. 2B).

Briefly:

Step 1: (2S)-5-[[(Z)—N,N′-bis(tert-butoxycarbonyl)carbamimidoyl]amino]-2-(tert-butoxy-carbonylamino)pentanoic acid

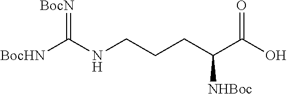

(2S)-2-amino-5-guanidino-pentanoic acid (17.4 g, 99.88 mmol, 1 eq) was dissolved in t-BuOH (250 mL) and H₂O (250 mL) then NaOH (14 g, 350.03 mmol, 3.50 eq) was added in portions at 0° C., then Boc2O (87.20 g, 399.54 mmol, 91.79 mL, 4 eq) was added in portions at 0° C. the mixture was stirred at 25° C. for 24 hr, The reaction mixture was evaporated to about ½ volume, then EtOAc(200 mL) was added, EtOAc phase was discarded, then carefully acidified with citric acid to pH 3 and was extracted with ethyl acetate (100 mL·2). The extract was dried with anhydrous sodium sulfate and evaporated to give the residue. The residue was dried in a vacuum oven to give (2S)-5-[[(Z)—N,N′-bis(tert-butoxycarbonyl)carbamimidoyl]amino]-2-(tert-butoxycarbonylamino)pentanoic acid (20 g, 42,15 mmol, 42.19% yield) as white solid.

Step 2: 2,2-dimethyl-6-nitro-4H-1,3-benzodioxine

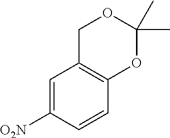

To a solution of 2-(hydroxymethyl)-4-nitro-phenol (9 g, 53.21 mmol, 1 eq) in Acetone (300 mL) was added TsOH.H₂O (2.02 g, 10.64 mmol, 0.2 eq) and 2,2-dimethoxypropane (11.64 g, 111.75 mmol, 13.69 mL, 2.1 eq) in one portion. Then the mixture was stirred at 40° C. for 8 hr. The mixture was quenched with NH₃.H₂O (3 mL), and then was evaporated to give the residue. The residue was purified by column chromatography (SiO₂, PE/EtOAc=:10/1 to 5:1) to obtain 2,2-dimethyl-6-nitro-4H-1,3-benzodioxine (9 g, 43.02 mmol, 80.85% yield) as yellow solid.

Step 3: 2,2-dimethyl-4H-1,3-benzodioxin-6-amine

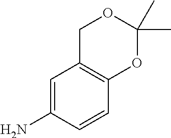

2,2-dimethyl-6-nitro-4H-1,3-benzodioxine (9 g, 43.02 mmol, 1 eq) was dissolved in MeOH (150 mL) and then Pd/C (1 g, 10% purity) was added in one portion, the mixture was stirred at 20° C. under H₂ at 15 psi for 2 hr. The reaction mixture was filtered to give the filtrate. The filtrate was evaporated to give the crude 2,2-dimethyl-4H-1,3-benzodioxin-6-amine (7.6 g, crude) as yellow solid.

Step 4: allyl N-(2,2-dimethyl-4H-1,3-benzodioxin-6-yl)carbamate

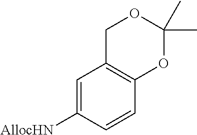

2,2-dimethyl-4H-1,3-benzodioxin-6-amine (7.5 g, 41.85 mmol, 1 eq) was dissolved in DCM (200 mL) and then pyridine (9.93 g, 125.55 mmol, 10.13 mL, 3 eq) was added in one portion, then allyl carbonochloridate (7.57 g, 62.77 mmol, 6.64 mL, 1.5 eq) was added in portions at 0° C. and stirred at 20° C. for 2 hr. The reaction mixture was quenched with saturated aq. NaHCO₃ (100 mL), then extracted with EtOAc(200 mL*2) and the solvent was evaporated to give the residue. The residue was purified by column chromatography (SiO2, PE/EA=8/1 to 5:1) to obtain allyl N-(2,2-dimethyl-4H-1,3-benzodioxin-6-yl)carbamate (10.5 g, 39.88 mmol, 95.30% yield) as yellow oil.

Step 5: allyl N-[4-hydroxy-3-(hydroxymethyl)phenyl]carbamate

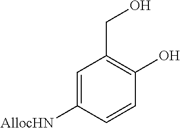

allyl N-(2,2-dimethyl-4H-1,3-benzodioxin-6-yl)carbamate (9 g, 34.18 mmol, 1 eq) was dissolved in AcOH (60 mL) and H₂O (30 mL), then the mixture was stirred at 70° C. for 0.5 hr. The reaction mixture was quenched with saturated NaHCO₃(400 mL), then was extracted with EtOAc (300 mL*2), the organic solvent was washed with water (200 mL) and then was washed with brine (200 mL), then the solvent was evaporated to give the residue. The residue was used to next step without further purification, allyl N-[4-hydroxy-3-(hydroxymethyl)-phenyl]carbamate (7.5 g, 33.60 mmol, 98.29% yield) was obtained as white solid.

Step 6: allyl N-(2-chloro-2-oxo-4H-1,3,2benzodioxaphosphinin-6-yl)carbamate

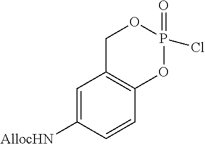

To a solution of allyl N-[4-hydroxy-3-(hydroxymethyl)phenyl]carbamate (3 g, 13.44 mmol, 1 eq) in THF (80 mL) was added TEA (4.08 g, 40.32 mmol, 5.61 mL, 3 eq) in one portion. Then POCl₃ (3.09 g, 20.16 mmol, 1.87 mL, 1.5 eq) was added dropwise at −40° C. and stirred at-40˜20° C. for 6 hr. The reaction mixture was filtered and the solvent was removed under reduced pressure using a high-vacuum pump. The residue was used to next step without further purification. allyl N-(2-chloro-2-oxo-4H-1,3,2benzodioxaphosphinin-6-yl)carbamate (3.5 g, crude) was obtained as yellow oil. (MS: [M+1]304.0).

Step 7: ally N-[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]carbamate

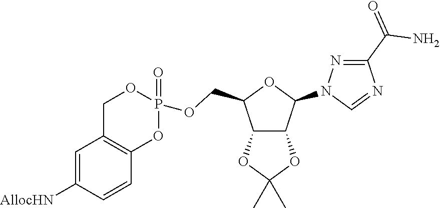

To a solution of 1-[(3aR,4R,6R,6aR)-6-(hydroxymethyl)-2,2-dimethyl-3a,4,6,6a-tetrahydrofuro[3,4-d][1,3]dioxol-4-yl]-1,2,4-triazole-3-carboxamide (3.5 g, 12.31 mmol, 1.10 eq.) in DCM (100 mL) was added allyl N-(2-chloro-2-oxo-4H-1,3,2benzodioxaphosphinin-6-yl)carbamate (3.4 g, 11.20 mmol, 1 eq) in one portion. Then N-methylimidazole (2.76 g, 33.59 mmol, 2.68 mL, 3 eq) was added dropwise at 20° C. and stirred at 20° C. for 1 hr, the reaction mixture was evaporated to give the residue. The residue was purified by reversed-phase column (0.1% HCOOH in 0%˜-70% water/CH₃CN) to give the desired peak, after lyophilization, got allyl N-[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2-benzodioxaphosphinin-6-yl]carbamate (3.0 g, 5.44 mmol, 48.58% yield) as yellow solid. (MS: [M+1]552.3).

Step 8: 1-[(3aR,4R,6R,6aR)-6-[(6-amino-2-oxo-4H-1,3,2benzodioxaphosphinin-2-yl)oxymethyl]-2,2-dimethyl-3a,4,6,6a-tetrahydrofuro[3,4-d][1,3]dioxol-4-yl]-1,2,4-triazole-3-carboxamide

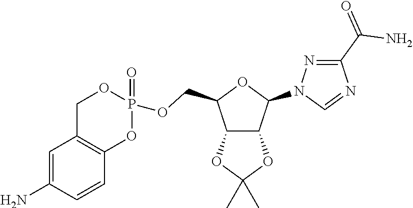 [SMILES: CC1(C)O[C@@H]2[C@H](O1)[C@@H](COP1(=O)OCc3cc(N)ccc3O1)O[C@H]2n1cnc(C(N)=O)n1]

To a solution of allyl N-[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo4H-1,3,2-benzo-dioxaphosphinin-6-yl]carbamate (800 mg, 1.45 mmol, 1 eq) in DCM (100 mL) was added 1,3-dimethylhexahydropyrimidine-2,4,6-trione (906.08 mg, 5.80 mmol, 4 eq) and Pd(PPh₃)₄ (251.46 mg, 217.61 μmol, 0.15 eq) in one portion. Then the mixture was stirred at 20° C. for 1 hr. The reaction mixture was evaporated to give the residue. The residue was used to next step without further purification. 1-[(3aR,4R,6R,6aR)-6-[(6-amino-2-oxo-4H-1,3,2benzodioxa-phosphinin-2-yl)oxymethyl]-2,2-dimethyl-3a,4,6,6a-tetrahydrofuro[3,4-d][1,3dioxol-4-yl]-1,2,4-triazole-3-carboxamide (660 mg, crude) was obtained as yellow solid. (MS: [M+1]468.3).

Step 9: tert-butyl (NZ)—N-[[[(4S)-5-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3,]dioxol-6-yl]methoxy]-2-oxo-4H-1,3,2-benzodioxaphosphinin-6-yl]amino]-4-(tert-butoxycarbonylamino)-5-oxo-pentyl]amino]-(tert-butoxycarbonylamino)methylene]carbamate

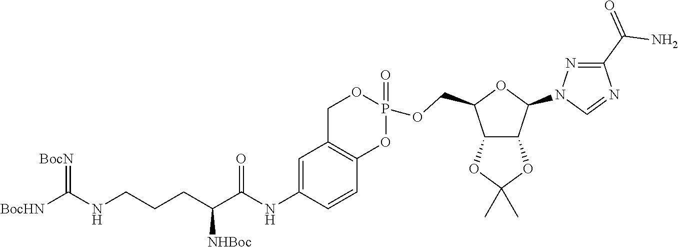

To a solution of 1-[(3aR,4R,6R,6aR)-6-[(6-amino-2-oxo-4H-1,3,2benzodioxaphosphinin-2-yl)oxymethyl]-2,2-dimethyl-3a,4,6,6a-tetrahydrofuro 3,4-d][1,3]dioxol-4-yl]-1,2,4-triazole-3-carboxamide (660 ng, 1.41 mmol, 1 eq) in THF (50 mL) was added (2S)-5-[[(Z)—N,N′-bis-(tert-butoxycarbonyl)carbaminidoyl]amino]-2-(tert-butoxycarbonvlamino)pentanoic acid (871.18 mg, 1.84 mmol, 1.3 eq) and HOBt (286.22 mg, 2.12 mmol, 1.5 eq) in one portion, then DCC (437.05 mg, 2.12 mmol, 1.5 eq) was added in one portion at 0° C. Then the mixture was stirred at 20° C. for 4 hr. The reaction mixture was evaporated to give the residue. The residue was purified by reversed-phase column (0.1% NH3.H2O in 0%˜70% water/CH₃CN) to give the desired peak, after lyophilization, got desired product. tert-butyl (NZ)—N-[[[(4S)-5-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2benzodioxaphosphinin-6-yl]amino]-4-(tert-butoxycarbonylamino)-5-oxo-pentyl]amino]-(-tert-butoxycarbonylamino)-methylene]carbamate (550 ng, 595.30 μmmol, 42.16% yield) as yellow solid. (MS: [M+1]924.4).

Step 10:

1-[(2R,3R,4S,5R)-5-[[6-[[(2S)-2-amino-5-guanidino-pentanoyl]amino]-2-oxo-4H-1,3,2-benzodioxaphosphinin-2-yl]oxymethyl]]-3,4-dihydroxy-tetrahydrofuran-2-yl]-1,2,4-triazole-3-carboxamide

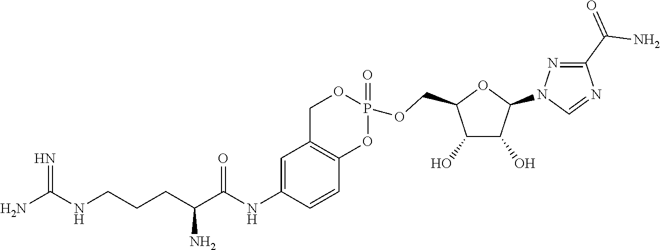 [SMILES: N=C(N)NCCC[C@H](N)C(=O)Nc1ccc2c(c1)COP(=O)(OC[C@H]1O[C@@H](n3cnc(C(N)=O)n3)[C@H](O)[C@@H]1O)O2]

tert-butyl (NZ)—N-[[[(4S)-5-[[2-[[(3aR,4R,6R,6aR)-4-(3-carbamoyl-1,2,4-triazol-1-yl)-2,2-dimethyl-3a,4,6,6a-tetrahydrofuro[3,4-d][1,3]dioxol-6-yl]methoxy]-2-oxo-4H-1,3,2 benzo-dioxaphosphinin-6-yl]amino]-4-(tert-butoxycarbonylamino)-5-oxo-pentyl]amino]-(tert-butoxycarbonylamino)methylene]carbamate (100 mg, 108.24 μmol, 1 eq) was dissolved in H₃PO₄ (1.68 g, 14.57 mmol, 1.00 mL, 85% purity, 134.63 eq), then the mixture was stirred at ° C. for 0.5 hr. Water (4 mL) was added to the mixture. The mixture was purified by reversed-phase column (0.1% HCOOH in 0%˜70% water/CH₃CN) to give the desired peak, after lyophilization, got 1-[(2R,3R,4S,5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide (16.7 mg, 26.04 μmol, 24.06% yield, 91% purity) as white solid. (MS: [M+1]584.3); ¹H NMR (McOD, 400 MHz): δ(ppm) 8.55 (s, 1H), 8.51 (s, 1H), 8.39 (s, 1H), 7.31-7.25 (m, 1H), 7.18-7.17 (m, 1H), 7.00-6.89 (m, 1H), 5.98 (s, 1H), 5.39-5.31 (m, 2H), 4.69-4.63 (m, 1H), 4.53-4.29 (m, 4H), 4.10-4.09 (m, 1H), 3.23-3.20 (m, 2H), 2.01-1.96 (m, 2H). 1.71-1.65 (m, 2H); ³¹P NMR (MeOD, 162 MHz): δ(ppm) −7.60, −8.07.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A compound of Formula I: 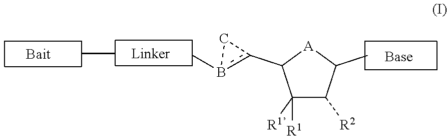
or a pharmaceutically acceptable salt thereof, wherein:
---- is an optional bond;
A is selected from the group consisting of CH₂, NH, O, and S;
B is selected from the group consisting of C, CH, CH₂, N, NH, O, and S;
C is selected from the group consisting of CH₂, NH, O, and S;
R¹ is selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{1′} is selected from the group consisting of OH, SH, NH₂, N₃, and halo;
R² is selected from the group consisting of OH, SH NH₂, N₃, and halo;
Base is selected from the group consisting of 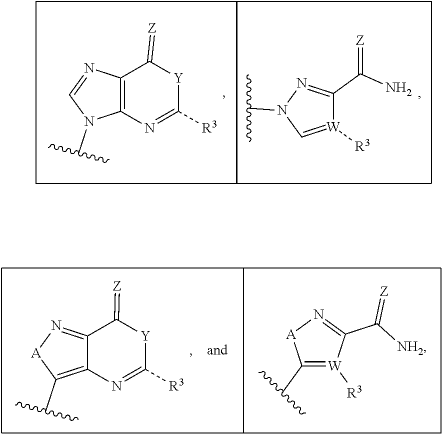
wherein:
Z is selected from the group consisting of O, NH, and S;
Y is selected from the group consisting of O, NH, and S;
W is selected from the group consisting of CH, CH₂, NH, and N;
R³ is selected from the group consisting of OH, SH, NH₂, N₃ and halo;
Linker is selected from the group consisting of 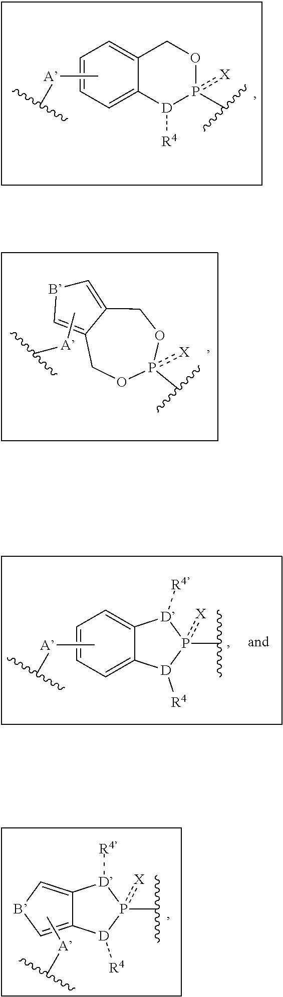
wherein:
A′ is independently, at each occurrence, selected from the group consisting of C₂, NH, O, S, CO₃, CO₂N, CO₂S, and CO₂N;
B′ is independently, at eachl occurrence, selected from the group consisting of CH₂, NH, O, and S;
D and D′ are each independently selected from the group consisting of O, N, NH, and S;
X is selected from the group consisting of O, NH, and S;
R⁴ and R^{4′} are each independently Selected from the group Consisting Of H, C₁₋₄ alkyl, C₂₋₄ alkenyl, C₂₋₄ vinyl, and C₃₋₄ allyl, all of which are optionally substituted with one, two, three, or four halo;
Bait is an oligomer comprising 1-5 units, which are independently, at each occurrence, selected from the group consisting of 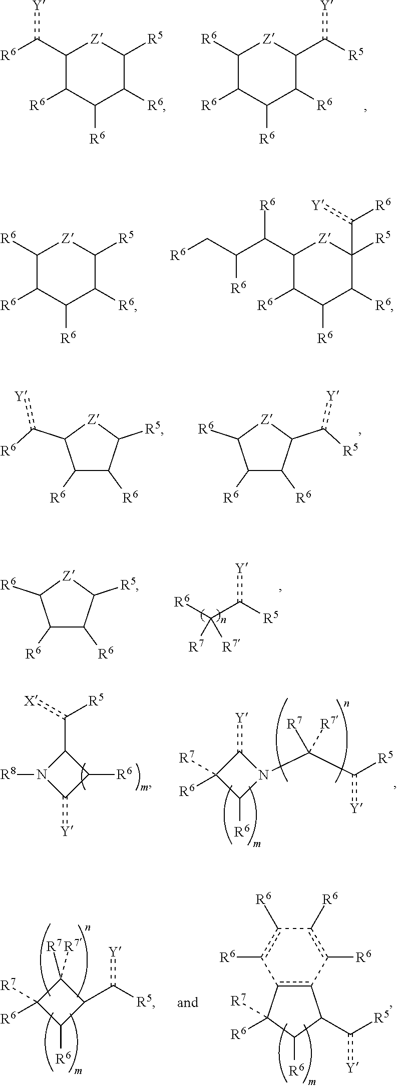
wherein;
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and a bond to A′;
R⁶ is independently, at each occurrence, selected from the group consisting of a bond to R⁵, H, ═O, ═NH, —R^{A}—(C═R^{B})—R^{C}—R^{A}—(C═R^{B})—R^{A}—R^{C}, R^{A}—(SO₂)—R^{D},—R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R^{7′} are each independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}(C═R^{B})—R^{C}, —R^{A}—(C═R^{B})—R^{A}—R^{C}, R^{A}—(SO₂)—R^{D}, —R^{A}—(SO₂)—R^{C}, —R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1, 2, or 3.

2. The compound of claim 1, wherein the compound of Formula I comprises a compound of Formula Ia: 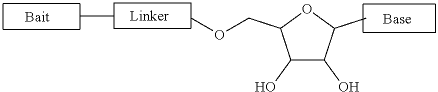
or a pharmaceutically acceptable salt thereof.

3. The compound of claim 1, wherein the compound of Fornmula I is a compound of Formula Ib: 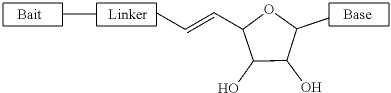
or a pharmaceutically acceptable salt thereof.

4. The compound of any one of claims 1-3, wherein Base comprises

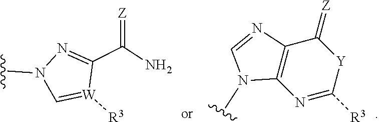

5. The compound of any one of claims 1-4, wherein Base comprises

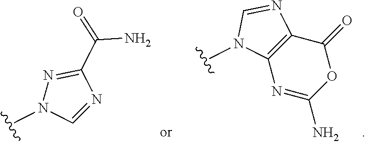

6. The compound of any one of claims 1-5, wherein Linker comprises

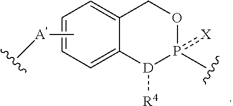

7. The compound of any one of claims 1-6, wherein Linker comprises

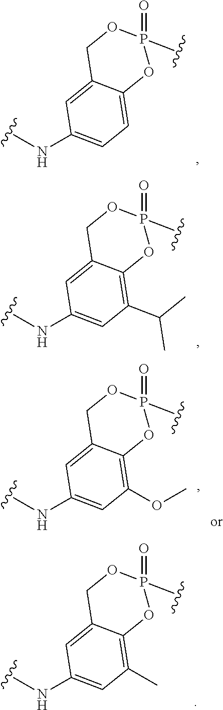

8. The compound of claim 1, wherein the compound of Formula I is selected from the group consisting of 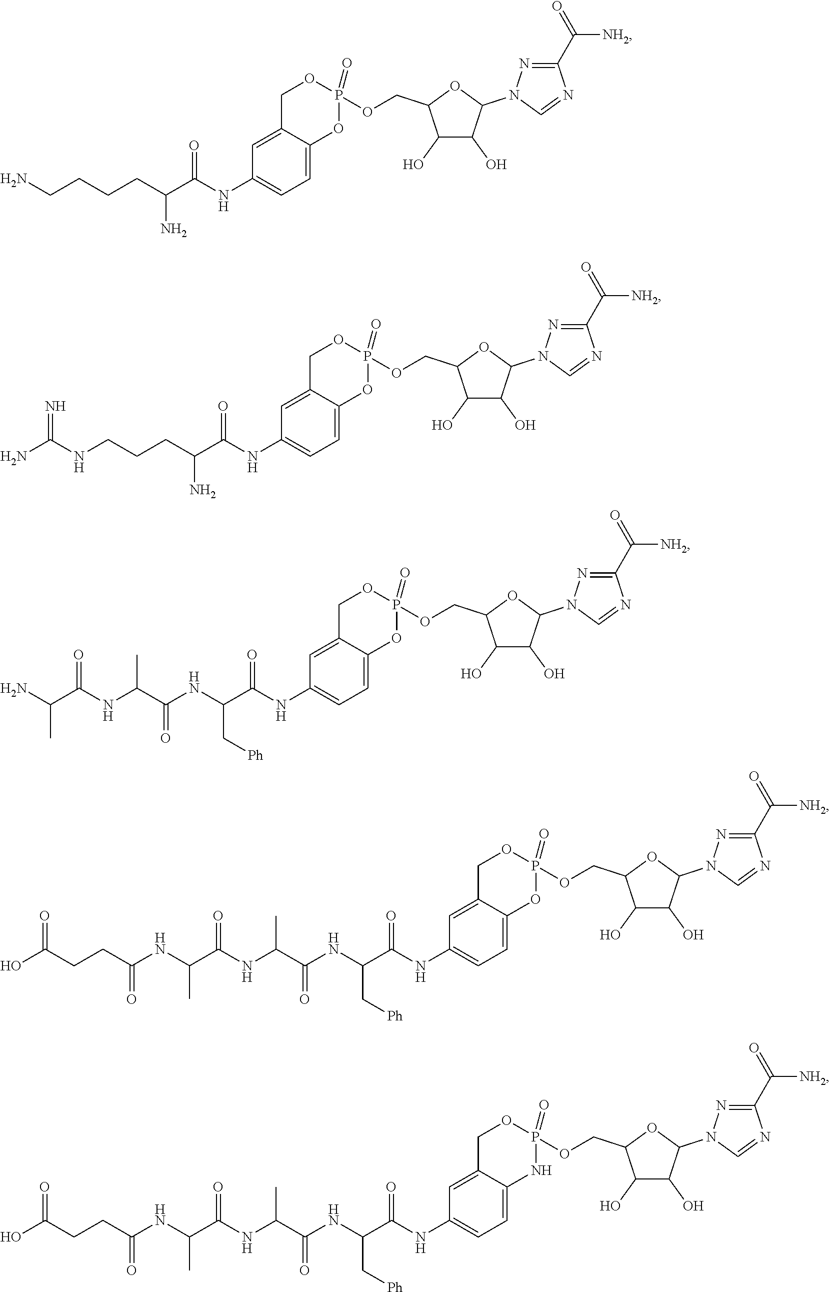 [SMILES: NCCCCC(N)C(=O)Nc1ccc2c(c1)COP(=O)(OCC1OC(n3cnc(C(N)=O)n3)C(O)C1O)O2 ; N=C(N)NCCCC(N)C(=O)Nc1ccc2c(c1)COP(=O)(OCC1OC(n3cnc(C(N)=O)n3)C(O)C1O)O2 ; CC(N)C(=O)NC(C)C(=O)NC(Cc1ccccc1)C(=O)Nc1ccc2c(c1)COP(=O)(OCC1OC(n3cnc(C(N)=O)n3)C(O)C1O)O2 ; CC(NC(=O)CCC(=O)O)C(=O)NC(C)C(=O)NC(Cc1ccccc1)C(=O)Nc1ccc2c(c1)COP(=O)(OCC1OC(n3cnc(C(N)=O)n3)C(O)C1O)O2 ; CC(NC(=O)CCC(=O)O)C(=O)NC(C)C(=O)NC(Cc1ccccc1)C(=O)Nc1ccc2c(c1)COP(=O)(OCC1OC(n3cnc(C(N)=O)n3)C(O)C1O)N2] 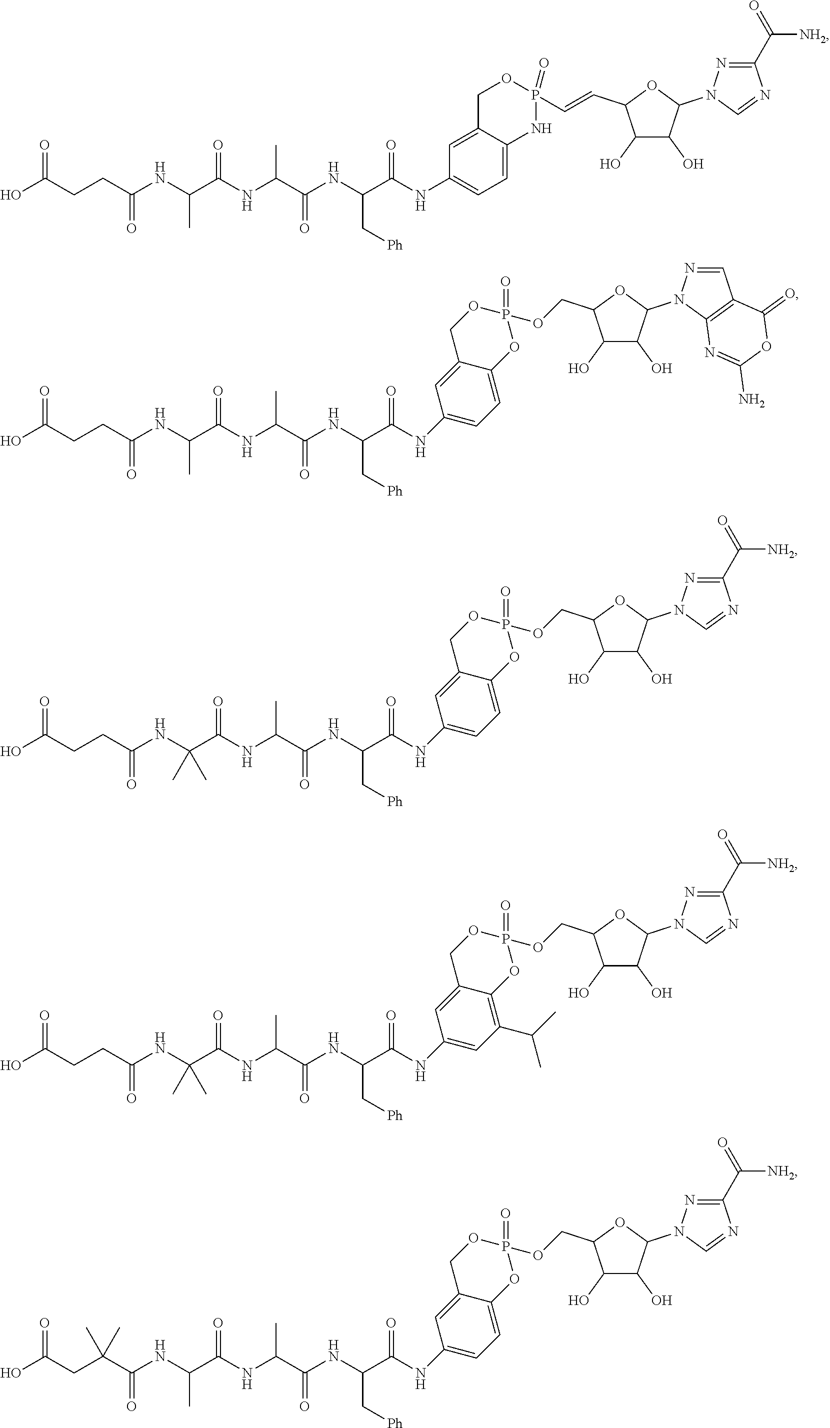 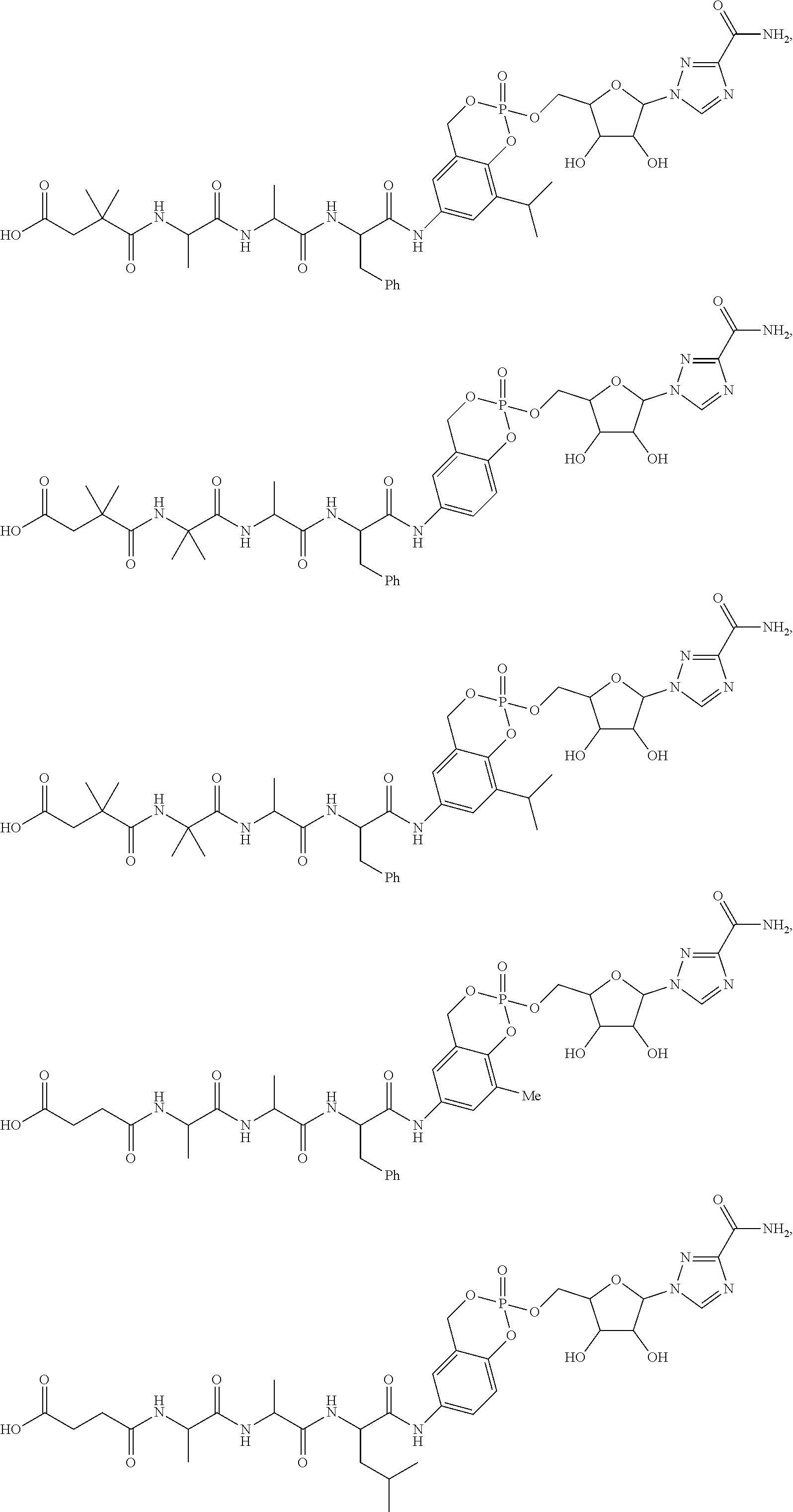 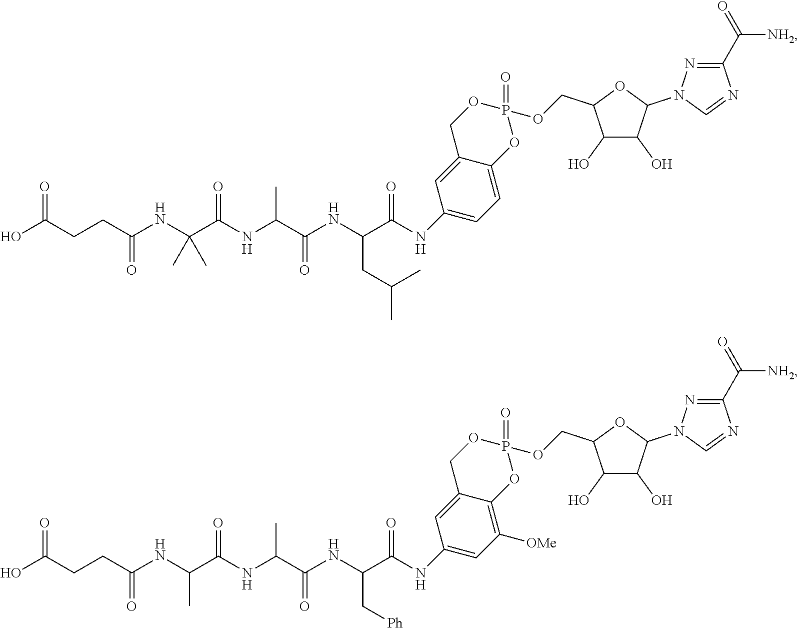 [SMILES: CC(C)CC(NC(=O)C(C)NC(=O)C(C)(C)NC(=O)CCC(=O)O)C(=O)Nc1ccc2c(c1)COP(=O)(OCC1OC(n3cnc(C(N)=O)n3)C(O)C1O)O2 ; COc1cc(NC(=O)C(Cc2ccccc2)NC(=O)C(C)NC(=O)C(C)NC(=O)CCC(=O)O)cc2c1OP(=O)(OCC1OC(n3cnc(C(N)=O)n3)C(O)C1O)OC2]
and a pharmaceutically acceptable salt thereof.

9. The compound of claim 1, wherein the compound of Formula I comprises a compound of Formula Ic: 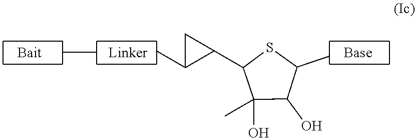
or a pharmaceutically acceptable salt thereof.

10. The compound of claim 9, wherein Base comprises

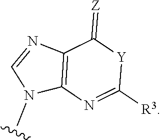

11. The compound of any one of claim 9 or 10, wherein Base comprises

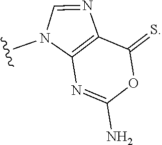

12. The compound of any one of claims 9-11, wherein Linker comprises

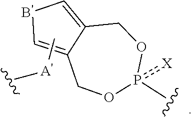

13. The compound of any one of claims 9-12, wherein Linker comprises

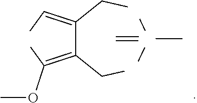

14. The compound of claim 9, wherein the compound of Formula Ic is selected from the group consisting of 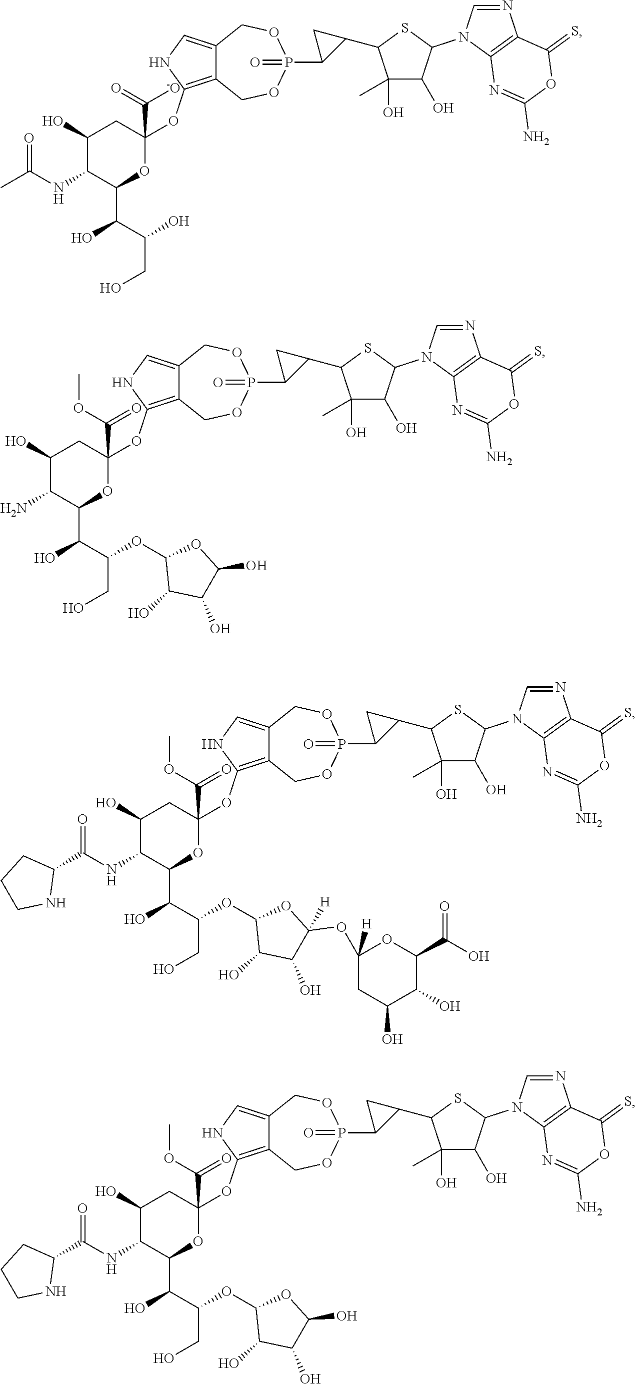 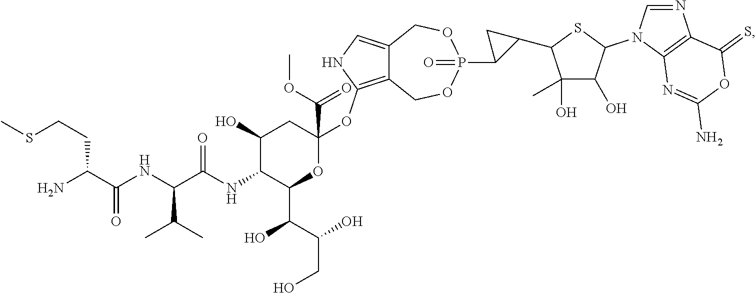
and a pharmaceutically acceptable salt thereof.

15. A compound of Formula II: 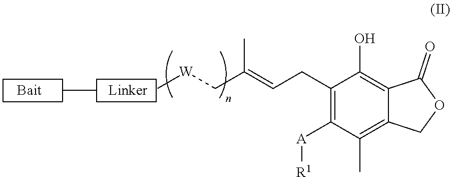
or a pharmaceutically acceptable salt thereof, wherein:
A is selected from the group consisting of CH₂, NH, O and S;
W is independently, at each occurrence, selected from the group consisting of CH, CH₂, NH, and N;
r is 1, 2, or 3;
Linker is selected from the group consisting of 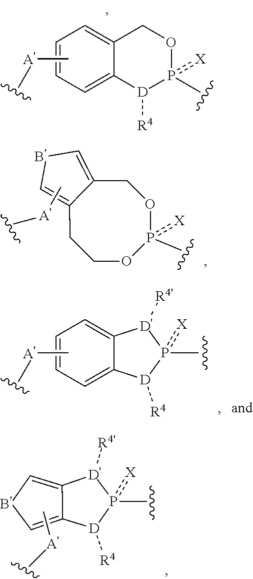
wherein:
A′ is independently, at each occurrence, selected from the group consisting of CH₂, NH, O, S, CO₃, CO₂N, CO₂S, and CO₂N;
B′ is independently, at each occurrence, selected from the group consisting of CH₂, NH, O, and S;
D and D′ are each independently selected from the group consisting of O, NH, and S;
X is selected from the group consisting of O, NH, and S; and
R⁴ and R^{4′} are each independently selected from the group consisting of H, C₁₋₄ alkyl, C₂₋₄ alkenyl, C₂₋₄ vinyl, and C₃₋₄ allyl, all of which are optionally substituted with one, two, three, or four halo.
Bait is an oligomer comprising 1-5 units, which are, independently at each occurrence selected from the group consisting of 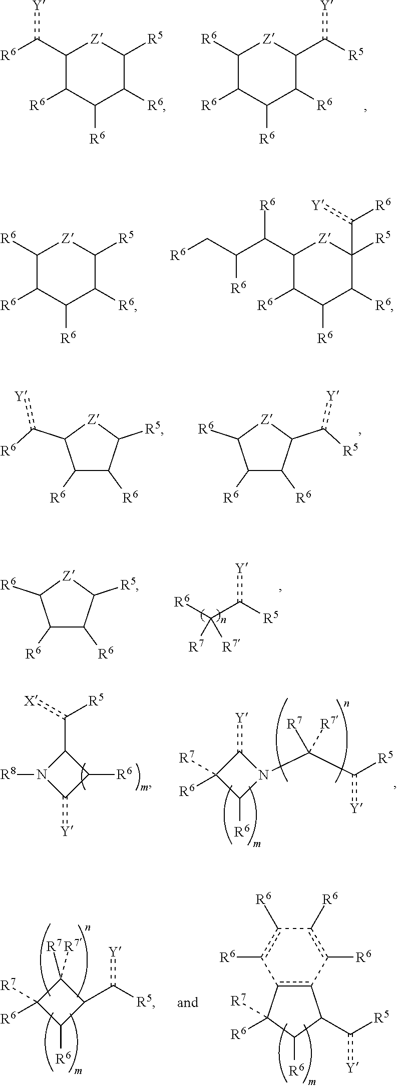
Wherein:
Z′ is independently, at each occurrence, selected from the group consisting of CH₂, NH, O, and S;
X′ and Y′ are independently, at each occurrence, selected from the group consisting of O, NH, and S;
R⁵ is independently, at each occurrence, selected from the group consisting of a bond to any R⁶, a bond to any R⁸, and a bond to A′;
R⁶ is independently, at each occurrence, selected from the group consisting of a bond to R⁵, H, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C}, and —R^{A}—(SO₂)—R^{A}—R^{C};
R^{A} is independently, at each occurrence, selected from the group consisting of CH₂, NH, O, and S;
R^{B} is independently, at each occurrence, selected from the group consisting of O, NH, and S;
R^{C} is independently, at each occurrence, selected from the group consisting of H, C₁₋₄ alkyl, C₂₋₄ alkenyl, C₂₋₄ vinyl, and C₃₋₄ allyl, all of which are optionally substituted with one, two, three, or four halo;
R^{D} is independently, at each occurrence, selected from the group consisting of OH, SH, NH₂, N₃, and halo;
R⁷ and R^{7′} are independently, at each occurrence, selected from the group consisting of H, NH₂, OH, C₆₋₁₀ aryl, 5-10 membered heteroaryl, C₁₋₄ alkyl, —(CH₂)₁₋₃—C₆₋₁₀ aryl, and —(CH₂)₁₋₃-5-10 membered heteroaryl; wherein aryl, heteroaryl, and alkyl are optionally substituted with one, two, or three substituents selected from the group consisting of halo, ═O, ═NH, ═S, —R^{A}—(C═R^{B})—R^{C}, —R^{A}—(C═R^{B})—R^{A}—R^{C}, R^{A}—(SO₂)—R^{D}, —R^{A}—(SO₂)—R^{C},—R^{A}—(SO₂)—R^{A}—R^{C}, C₁₋₄ alkyl, C₂₋₄ alkenyl, C₂₋₄ vinyl, and C₃₋₄ allyl;
R⁸ is independently, at each occurrence, a bond to R⁵;
m is 0, 1, 2, 3, or 4; and
n is 0, 1,2, or 3.

16. The compound of claim 15, wherein the compound of Formula II comprises a compound of Formula IIa:

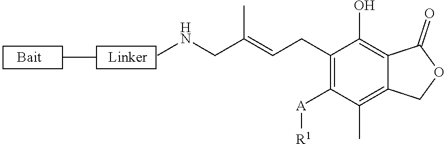

17. The compound claim 15 or 16, wherein Linker comprises

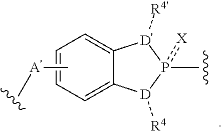

18. The compound of any one of claims 15-17, wherein Linker comprises

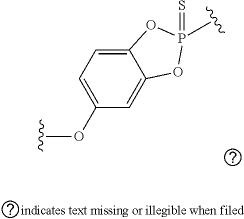

19. The compound of any one of claims 15-18, wherein the compound of Formula I is selected from the group consisting of 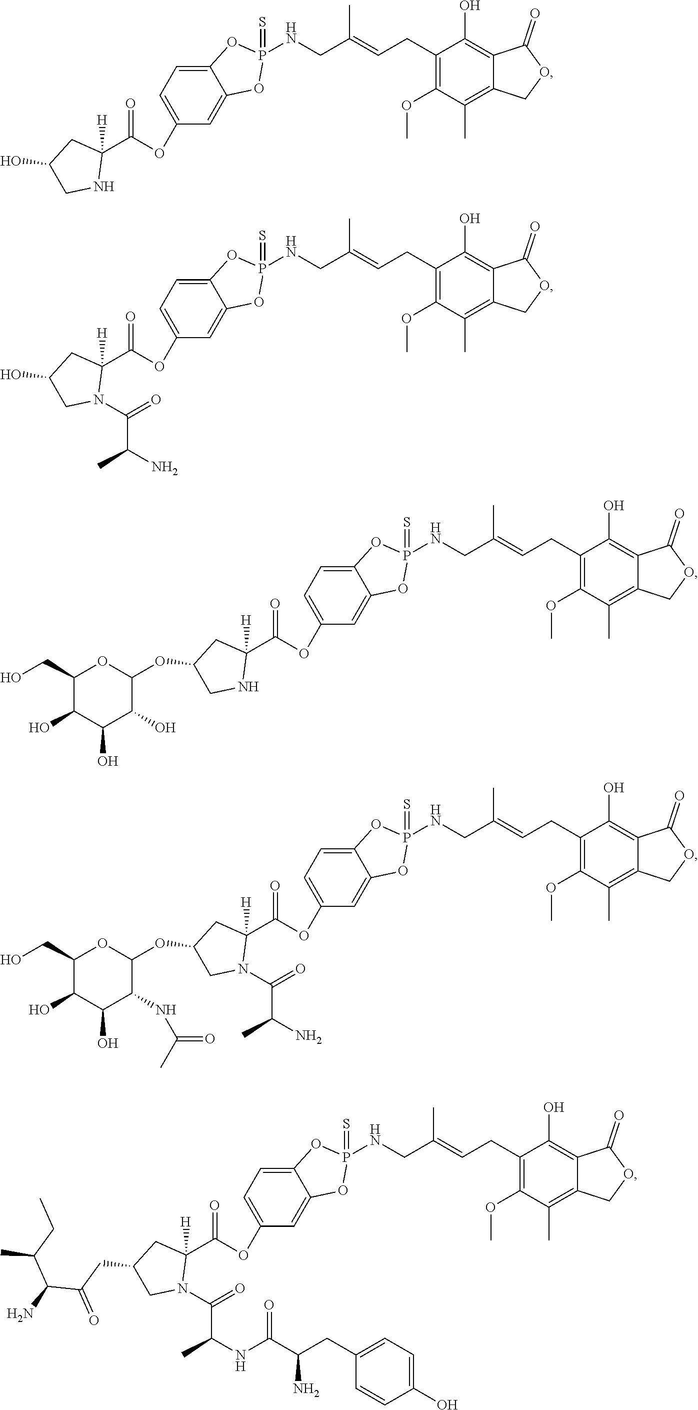
and a pharmaceutically acceptable salt thereof.

20. A pharmaceutical formulation comprising a CSII compound of any one of claims 1-19, and a pharmaceutically acceptable carrier.

21. The pharmaceutical formulation of claim 20, further comprising an immunomodulatory or anti-proliferative compound different than the CSII compound.

22. A method of treating proliferative disorder in a patient, comprising administering to the patient an amount of the formulation of claim 20 or 21 effective to reduce or inhibit a symptom of the proliferative disorder.

23. A method of treating a proliferative disorder in a patient, comprising administering to the patient a CSII compound of any one of claims 1-19 in a pharmaceutically acceptable carrier, in an amount effective to reduce or inhibit at least one symptom of the proliferative disorder.

24. A method of synthesizing a CSII compound of any one of claims 1-19, comprising carrying out the protocol of scheme 1 as set forth in FIG. 2A.

25. A method of synthesizing a CSII compound of any one of claims 1-19, comprising carring out the protocol of scheme 1 as set forth in FIG. 2B.

26. A method of synthesizing 1-[(2R,3R,4S,5R)-5-[[6-[[(2S)-2,6-diaminohexanoyl]-amino]-2-oxo-4H-1,3,2benzodioxaphosphinin-2-yl]oxymethyl]-3,4-dihydroxyetrahydrofuran-2-yl]-1,2,4-triazole-3-carboxamide, comprising carrying out the protocol set forth in EXAMPLE 1 and FIG. 2A.

27. A method of synthesizing 1-[(2R,3R,4S,5R)-5-[[6-[[(2S)-2-amino-5-guanidino-pentanoyl]-amino]-2-oxo-4H-1,3,2benzodioxaphosphinin-2-yl]oxymethyl]-3,4-dihydroxy-tetrahydro-furan-2-yl]-1,2,4-triazole-3-carboxamide, comprising carrying out the protocol set forth in EXAMPLE 2 and FIG. 2B

28. A method of modulating the activity of an immune cell, comprising contacting the cell with a CSII of any one of claims 1-19 effective to reduce or inhibiting the activity of inosine-5′-monophosphate dehydrogenase (IMPDH).
